(19) United States
(12) Patent Application Publication
(10) Pub. No.: US 2025/0232436 A1
(43) Pub. Date: Jul. 17, 2025

(54) DETERMINING CHANGES IN PUPIL MORPHOLOGY DURING AN OPHTHALMIC SURGICAL PROCEDURE

(71) Applicant: REGENTS OF THE UNIVERSITY OF MICHIGAN, Ann Arbor, MI (US)

(72) Inventors: Nambi Nallasamy, Ann Arbor, MI (US); Bradford Tannen, Superior Township, MI (US); Shahzad Mian, Superior Township, MI (US)

(21) Appl. No.: 19/019,779

(22) Filed: Jan. 14, 2025

Related U.S. Application Data
(60) Provisional application No. 63/621,208, filed on Jan. 16, 2024.

(51) Int. Cl. G06T 7/00; G06T 7/11; G06T 7/62; G06T 7/64; G16H 50/20

(52) U.S. Cl. CPC G06T 7/0012; G06T 7/11; G06T 7/62; G06T 7/64; G16H 50/20; G06T 2207/10016; G06T 2207/10024; G06T 2207/20064; G06T 2207/20081; G06T 2207/20084; G06T 2207/30041

(57) ABSTRACT

System and methods for determining pupil morphology during an ophthalmic surgical procedure. The system may receive surgical video data of an ophthalmic surgical procedure. The surgical video data may include images of at least a portion of an eye. The system may provide the surgical video data to an adaptive wavelet tensor feature extraction machine learning (ML) model to generate preprocessed image data, including preprocessed images with extracted features associated with anatomical components of the eye. The system may provide the preprocessed image data to an anatomy segmentation ML model to generate segmented image data and segmentation masks. The system may provide the segmented image data and segmentations masks to an obstruction classifier ML model to determine a size of a pupil of the eye in the segmented images. The system may generate a notification based upon the size of the pupil, and provide the notification to a user device.

### CROSS-REFERENCE TO RELATED APPLICATION

This application claims priority to and the benefit of the filing date of provisional U.S. Patent Application No. 63/621,208, entitled “DETERMINING CHANGES IN PUPIL MORPHOLOGY DURING AN OPHTHALMIC SURGICAL PROCEDURE” and filed on Jan. 16, 2024, the entire contents of which is hereby expressly incorporated herein by reference.

### GOVERNMENT LICENSE RIGHTS

This invention was made with government support under EY022299 awarded by the National Institutes of Health. The government has certain rights in the invention.

### FIELD OF THE DISCLOSURE

The present disclosure generally relates to systems and methods for analyzing surgical images, and more particularly, to systems and methods for using machine learning to determine changes in pupil morphology during an ophthalmic surgical procedure.

### BACKGROUND

Cataract surgery remains the only definitive treatment for visually significant cataracts, which are a major cause of preventable blindness worldwide. Because the cataract is anatomically located behind the iris, the majority of active surgical maneuvers in cataract surgery occur within the boundaries of the pupil. As such, successful performance of cataract surgery relies on stable dilation of the pupil. Although the pupil is pharmacologically dilated during surgery, pupillary instability is a major risk factor for cataract surgery complications. For instance, an adequately dilated pupil is required for safe cataract extraction during cataract surgery.

Automated pupil recognition from ophthalmic surgical images/videos can assist surgeons in detecting risk factors for pupillary instability prior to the development of surgical complications, and can also allow surgeons to understand intraoperative changes in pupil morphology (e.g., eccentricity, convex hull, major axis length, minor axis length, contour path length, etc.). However, surgical illumination variations, surgical instrument obstruction of the eye during the surgical procedure, and/or lens material hydration during cataract surgery can limit the accuracy of pupil recognition. For example, a typical cataract surgery can be broken into multiple surgical phases: paracentesis, medication injection, viscoelastic insertion, main wound, capsulorrhexis initiation, capsulorrhexis formation, hydrodissection, phacoemulsification, cortical removal, lens insertion, viscoelastic removal, and wound closure. These surgical phases involve different instrumentation and result in varying appearances of the pupil intraoperatively, e.g., a keratome is used to create the main wound, a phacoemulsification handpiece is used to break up the cataractous lens, and an irrigation-aspiration handpiece is used to remove cortical material and viscoelastic from the eye. In addition, changes in the cataractous lens and its eventual replacement with an intraocular lens implant lead to substantial changes in the appearance of the pupil region during the course of cataract surgery.

Methods for pupil recognition can be roughly divided into two categories: conventional computer vision-based methods and deep learning-based methods. While deep learning models may be superior in pupil recognition accuracy compared to conventional computer vision-based methods, there still exists a gap in their performance, owing to several inefficiencies. One such shortcoming arises from the difficulty in effectively processing images acquired from high-variation environments, such intraoperative pupil images in cataract surgery as just described.

Pupillary changes related to cataract surgery primarily focus on changes between preoperative and postoperative measurements of the pupil, ignoring intraoperative changes in pupil size. One reason may be that surgery-related pupillary changes often rely on specialized pupillometry hardware not suitable for intraoperative use, requiring intraoperative pupil measurements to be obtained manually and at very few time points during the surgery.

The conventional techniques for determining pupil morphology may include additional ineffectiveness, inefficiencies, encumbrances, and/or other drawbacks.

Therefore, there is an opportunity to provide improvements in, and a need for systems and methods for, determining pupil morphology during an ophthalmic surgical procedure in an automated, timely manner which avoid the shortcomings and inconsistencies of the present techniques.

### BRIEF SUMMARY

The present embodiments may relate to, inter alia, systems and methods for determining and/or predicting pupil morphology during an ophthalmic surgical procedure.

In one aspect, a system for determining pupil morphology during an ophthalmic surgical procedure. The system may include one or more processors, and one or more non-transitory memories storing processor-executable instructions that, when executed by the one or more processors, cause the system to: (i) receive surgical video data of at least a portion of an ophthalmic surgical procedure, wherein the surgical video data may include one or more images of at least a portion of an eye; (ii) provide the surgical video data to an adaptive wavelet tensor feature extraction machine learning model trained using feature extraction training data to generate preprocessed image data which may include one or more preprocessed images including extracted features associated with anatomical components of the eye; (iii) provide the preprocessed image data to an anatomy segmentation machine learning model trained using anatomy segmentation training data to generate: (a) segmented image data which may include one or more segmented images wherein the anatomical components are segmented; and (b) one or more segmentation masks associated with the anatomical components; (iv) provide the segmented image data and the one or more segmentation masks to an obstruction classifier machine learning model trained using obstruction classifier training data to determine a size of a pupil of the eye in the one or more segmented images, the determination of the size of the pupil may include: (a) determining one or more obstructed portions of the one or more segmented images using the one or more segmentation masks; (b) determining the size of the pupil using unobstructed portions of the pupil in the one or more segmented images; (c) determining the size of a limbus of the eye using unobstructed portions of the limbus in the one or more segmented images; and (d) normalizing the size of the pupil using the size of the limbus; (v) generate a notification based upon the size of the pupil; and (vi) provide the notification to a user device. The system may include additional, less, or alternate functionality, including that discussed elsewhere herein.

In one embodiment of the system, the ophthalmic surgical procedure may be selected from the group consisting of a cataract surgery, a vitreoretinal surgery, or a corneal surgery.

In another embodiment of the system, the one or more images may include color images.

In yet another embodiment of the system, the anatomical components may include one or more of the pupil, the limbus, a sclera, or a palpebral fissure.

In still yet another embodiment of the system, to generate the preprocessed image data, the adaptive wavelet tensor feature extraction machine learning model may be further trained to generate a third-order tensor of the pupil based upon a color image of the pupil of the surgical video data.

In another embodiment of the system, the anatomy segmentation machine learning model includes a convolutional neural network.

In yet another embodiment of the system the one or more segmentation masks may be associated with one or more of a palpebral fissure, the limbus, or the pupil.

In still yet another embodiment of the system, the one or more obstructed portions of the one or more segmented images may include obstructions from one or more of a surgical instrument, an eyelid, or a surgical drape.

In another embodiment of the system, the notification may indicate one or more of the size of the pupil, a prediction of pupil morphology, a recommendation of a pupil expansion device, a billing code associated with the ophthalmic surgical procedure, or a prediction associated with intraoperative floppy iris syndrome.

In yet another embodiment of the system, the size of the pupil may be normalized based upon magnification of the pupil in the one or more segmented images.

In another embodiment of the system, the size of the pupil includes one or more of an area of the pupil, an eccentricity of the pupil, a convex hull of the pupil, a major axis length of the pupil, a minor axis length of the pupil, or a contour path length of the pupil.

In still yet another embodiment of the system, the surgical video data may include at least a first phase and a second phase of the ophthalmic surgical procedure, and the system may further include instructions that, when executed by the one or more processors, cause the system to (i) determine the size of the pupil of the eye during the first phase of the ophthalmic surgical procedure; and one or more of: (ii)(a) determine the size of the pupil of the eye during the second phase of the ophthalmic surgical procedure, and (ii)(b) determine a change in the size of the pupil between the first phase and the second phase of the ophthalmic surgical procedure based upon comparing the size of the pupil during the first phase and the second phase of the ophthalmic surgical procedure, wherein generating the notification is further based upon the change in the size of the pupil, or (iii) predict the size of the pupil of the eye during the second phase of the ophthalmic surgical procedure based upon the size of the pupil during the first phase of the ophthalmic surgical procedure, wherein generating the notification is further based upon the prediction of the size of the pupil.

In another embodiment of the system, a phase of the ophthalmic surgical procedure may be selected from the group consisting of paracentesis, medication injection, viscoelastic insertion, main wound, capsulorrhexis initiation, capsulorrhexis formation, hydrodissection, phacoemulsification, cortical removal, lens insertion, viscoelastic removal, and wound closure.

In another aspect, a computer-implemented method for determining pupil morphology during an ophthalmic surgical procedure. The computer-implemented method may include: (i) receiving, by one or more processors, surgical video data of at least a portion of an ophthalmic surgical procedure, wherein the surgical video data may include one or more images of at least a portion of an eye; (ii) providing, by the one or more processors, the surgical video data to an adaptive wavelet tensor feature extraction machine learning model trained using feature extraction training data to generate preprocessed image data which may include one or more preprocessed images including extracted features associated with anatomical components of the eye; (iii) providing, by the one or more processors, the preprocessed image data to an anatomy segmentation machine learning model trained using anatomy segmentation training data to generate: (a) segmented image data which may include one or more segmented images wherein the anatomical components are segmented; and (b) one or more segmentation masks associated with the anatomical components; (iv) providing, by the one or more processors, the segmented image data and the one or more segmentation masks to an obstruction classifier machine learning model trained using obstruction classifier training data to determine a size of a pupil of the eye in the one or more segmented images, the determination of the size of the pupil may include: (a) determining, by the one or more processors, one or more obstructed portions of the one or more segmented images using the one or more segmentation masks; (b) determining, by the one or more processors, the size of the pupil using unobstructed portions of the pupil in the one or more segmented images; (c) determining, by the one or more processors, the size of a limbus of the eye using unobstructed portions of the limbus in the one or more segmented images; and (d) normalizing, by the one or more processors, the size of the pupil using the size of the limbus; (v) generating, by the one or more processors, a notification based upon the size of the pupil; and (vi) providing, by the one or more processors, the notification to a user device. The method may include additional, less, or alternate functionality or actions, including those discussed elsewhere herein.

In another aspect, a non-transitory computer-readable storage medium storing executable instructions that, when executed by a processor, cause a computer to at least: (i) receive surgical video data of at least a portion of an ophthalmic surgical procedure, wherein the surgical video data may include one or more images of at least a portion of an eye; (ii) provide the surgical video data to an adaptive wavelet tensor feature extraction machine learning model trained using feature extraction training data to generate preprocessed image data which may include one or more preprocessed images including extracted features associated with anatomical components of the eye; (iii) provide the preprocessed image data to an anatomy segmentation machine learning model trained using anatomy segmentation training data to generate: (a) segmented image data which may include one or more segmented images wherein the anatomical components are segmented; and (b) one or more segmentation masks associated with the anatomical components; (iv) provide the segmented image data and the one or more segmentation masks to an obstruction classifier machine learning model trained using obstruction classifier training data to determine a size of a pupil of the eye in the one or more segmented images, the determination of the size of the pupil may include: (a) determining one or more obstructed portions of the one or more segmented images using the one or more segmentation masks; (b) determining the size of the pupil using unobstructed portions of the pupil in the one or more segmented images; (c) determining the size of a limbus of the eye using unobstructed portions of the limbus in the one or more segmented images; and (d) normalizing the size of the pupil using the size of the limbus; (v) generate a notification based upon the size of the pupil; and (vi) provide the notification to a user device. The instructions may direct additional, less, or alternate functionality, including that discussed elsewhere herein.

In accordance with the above, and with the disclosure herein, the present disclosure includes improvements in computer functionality and/or improvements to other technologies at least because the claims recite, e.g., one or more machine learning (ML) models configured to receive surgical video data and determine the size of a pupil, and/or changes in pupil morphology, during an ophthalmic surgical procedure. An adaptive wavelet tensor feature extraction (AWTFE) ML model may receive the surgical video data to generate preprocessed image data including images wherein anatomical components of the eye are extracted, and background noise and irrelevant structures are diminished. Using the adaptive wavelet tensor-based method for feature extraction eliminates noise from the surgical video data images and more effectively extracts features of the eye (e.g., the anatomical components), overcoming limitations of other pupil recognition systems due to surgical illumination variations, obstruction if the eye by surgical instrument, lens material hydration, etc., to improve the accuracy of pupil recognition.

The preprocessed image data may be provided to an anatomy segmentation ML model to generate segmented image data including segmented images wherein the anatomical components are segmented. The anatomy segmentation ML model may generate segmentation masks of the anatomical components from the preprocessing image data. Preprocessing the surgical video data using the AWTFE ML model to extract features of the anatomical components in the preprocessed image data improves the performance of the anatomy segmentation ML model, e.g., improving the ability of the anatomy segmentation ML model to successfully segment the anatomical features in the segmented image data.

The segmented image data and segmentation masks may be provided to an obstruction classifier ML model to generate the size of the pupil. The segmentation of the anatomical features such as the pupil and limbus improve performance of the obstruction classifier ML model, e.g., by facilitating more accurate detection of obstructions of the anatomical components, such the pupil, which may otherwise affect the ability to determine the size of the pupil had the obstructions not been detected. Moreover, the segmentation masks may be used to more accurately predict the size of the pupil or other anatomical components, whether obscured or unobscured. These benefits and advantages are apparent when determining the size of the pupil by the obstruction classifier ML model when (i) determining one or more obstructed portions of the segmented images using the segmentation masks; (ii) determining the size of the pupil using unobstructed portions of the pupil in the segmented images; (iii) determining the size of a limbus of the eye using unobstructed portions of the limbus in the one or more segmented images; and (iv) normalizing the size of the pupil using the size of the limbus. For example, if the obstruction classifier ML model determines the pupil is unobstructed in the segmented images (the segmentation of the pupil improving the ability of the obstruction classifier ML model to detect the pupil based upon its segmented edges, contours, etc.), the obstruction classifier ML model may determine the size of the pupil based upon the size of the segmentation mask associated with the pupil. If the pupil is obstructed, the obstruction classifier ML model may generate an ellipse based upon the unobstructed regions of the pupil, and determine the size of the pupil based upon the ellipse. The obstruction classifier ML model may execute a similar process to determine the size of the limbus, which is used to normalize the size of the pupil, as the size may otherwise be inaccurate due to cropping of the pupil in the image data (e.g., due to decentration of the field of view of the camera recording the surgical video), variations in magnification and/or ocular rotation of the camera, etc.

The disclosed systems, methods and techniques may determine changes in the size of the pupil, e.g., throughout various phases of the surgery, alleviating the need for a surgical team to manually measure the size of the cataract during a surgery. The determination(s) of the size of the pupil and/or change in size of the pupil may occur in real-time, such that notifications associated with the (change in) pupil size may likewise be provided in real-time, e.g., on a surgical display. In one example, this allows the surgical team to be made aware of pupillary instability or other risk factors for surgical complications, such as an inadequately dilated pupil which may prevent safe extraction of a cataract during surgery. In one example, the disclosed systems, methods and techniques may be used as part of decision support and/or training systems for early-stage surgeons, e.g., to alert the surgeon of the need to use a pupil expansion device. All of these improvements and advantages allow the surgical team to focus on the surgery, and receive accurate and timely notifications associated with the (change in) size of the pupil of a surgical patient. As cataract surgery is one of the most common surgical procedures, this may improve the outcome and safety of hundreds, if not thousands, of surgical procedures and patients respectively.

In various aspects, a surgical system may be updated to implement the techniques associated with determining changes in pupil morphology during an ophthalmic surgical procedure in order to improve the functionality of the surgical system itself. That is, the present disclosure describes improvements in the functioning of the computer itself or “any other technology or technical field” because the quality of the surgical video, as used by a surgical system to determine the size of a pupil, may be improved, enhanced, or otherwise optimized, which not only improves the underlying surgical system by allowing more efficient and accurate detection of anatomical components of the eye, among other things, associated with determining the (change in) size of the pupil, but also improves the execution speed and/or efficiency of the determination of the (change in) pupil size by the surgical system when making such determinations in real-time and during the ophthalmic surgical procedure. This improves over the prior art at least because system or platforms, which would otherwise be required to implement multiple, different, and/or less effective means to determine pupil size (e.g., conventional computer vision-based methods and deep learning-based methods suffering from the aforementioned shortcomings and defects) before or after a surgical procedure, may now implement the disclosed systems and methods in real-time during the ophthalmic surgical procedure.

The present disclosure includes specific features other than that which is well-understood, routine, conventional activity in the field, and/or otherwise adds unconventional steps that confine the disclosure to a particular useful application, e.g., systems and methods for determining pupil morphology during an ophthalmic surgical procedure.

Additional, alternate and/or fewer actions, steps, features and/or functionality may be included in an aspect and/or embodiments, including those described elsewhere herein.

### BRIEF DESCRIPTION OF THE DRAWINGS

The figures described below depict various aspects of the system and methods disclosed therein. It should be understood that each figure depicts one embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

There are shown in the drawings arrangements which are presently discussed, it being understood, however, that the present aspects are not limited to the precise arrangements and instrumentalities shown, wherein:

FIG. 1 depicts a block diagram of an exemplary computing environment in which methods and system for determining pupil morphology during an ophthalmic surgical procedure are implemented, according to one embodiment;

FIG. 2 depicts a block diagram of an exemplary computing device in which computer-implemented methods and systems for determining pupil morphology during an ophthalmic surgical procedure are implemented, according to one embodiment;

FIG. 3 depicts a combined block and logic diagram for training machine learning models to determine pupil morphology during an ophthalmic surgical procedure, according to one embodiment;

FIG. 4A depicts a block diagram of an exemplary high-level system flow for determining pupil morphology during an ophthalmic surgical procedure, according to one embodiment;

FIG. 4B depicts exemplary ophthalmic surgical procedure images and associated feature-extracted images, according to one embodiment;

FIG. 4C depicts an exemplary ophthalmic surgical procedure image, exemplary red, green, and blue channel wavelets images, and an exemplary third-order wavelet subband tensor, according to one embodiment;

FIG. 4D depicts exemplary ophthalmic surgical procedure images and associated pupil segmentation masks, according to one embodiment;

FIG. 4E depicts exemplary images associated with generating the size of an obscured pupil, according to one embodiment; and

FIG. 5 depicts a flow diagram of an exemplary computer-implemented method for determining pupil morphology during an ophthalmic surgical procedure, according to one embodiment.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

### Overview

The computer systems and methods disclosed herein generally relate to, inter alia, determining pupil morphology during an ophthalmic surgical procedure. Using the disclosed techniques, pupil morphology may be determined during an ophthalmic surgical procedure in an automated fashion, which may include determining pupil morphology in real-time. One or more notifications associated with the size of the pupil may be provided to a user device, such a surgical system and/or display. The techniques provide a surgeon the ability to easily and quickly ascertain the size of the pupil during one or more phases of the surgery without having to manually measure the size and/or take attention away from the surgical patient. Further, the disclosed techniques improve pupil recognition by preprocessing the surgical video data to extract relevant anatomical components of the eye in the images, generating segmented images and segmentation masks of the anatomical components, and determining a normalized size of the pupil, which alleviate inefficiencies and inaccuracies due to illumination variations in the images, surgical instrument obstruction of the eye during a surgical procedure, among other things.

The disclosed systems and methods determine pupil morphology during an ophthalmic surgical procedure. In one embodiment, the system may receive the surgical video data which includes one or more images of at least a portion of an eye during the ophthalmic surgical procedure. The system may provide the surgical video data to an AWTFE ML model trained using feature extraction training data to generate preprocessed image data comprising one or more preprocessed images including extracted features associated with anatomical components of the eye. The system may provide the preprocessed image data to an anatomy segmentation ML model trained using anatomy segmentation training data to generate segmented image data comprising one or more segmented images wherein the anatomical components are segmented, and one or more segmentation masks associated with the anatomical components. The system may provide the segmented image data and the one or more segmentation masks to an obstruction classifier ML model trained using obstruction classifier training data to determine a size of a pupil of the eye in the one or more segmented images. The determination of the size of the pupil may include (i) determining one or more obstructed portions of the one or more segmented images using the one or more segmentation masks; (ii) determining the size of the pupil using unobstructed portions of the pupil in the one or more segmented images; (iii) determining the size of a limbus of the eye using unobstructed portions of the limbus in the one or more segmented images; and (iv) normalizing the size of the pupil using the size of the limbus. The system may generate a notification based upon the size of the pupil, and provide the notification to a user device.

As used herein, the terms video data and/or image data (e.g., surgical video data, preprocessed image data, segmented image data, etc.) refer to data comprising one or more images (e.g., images of a surgical procedure, preprocessed images including extracted features, segmented images, etc.). Accordingly, at times the terms “video data,” “image data,” and the like may be used interchangeably with the terms “image,” “images,” and the like.

### Exemplary Computing Environment

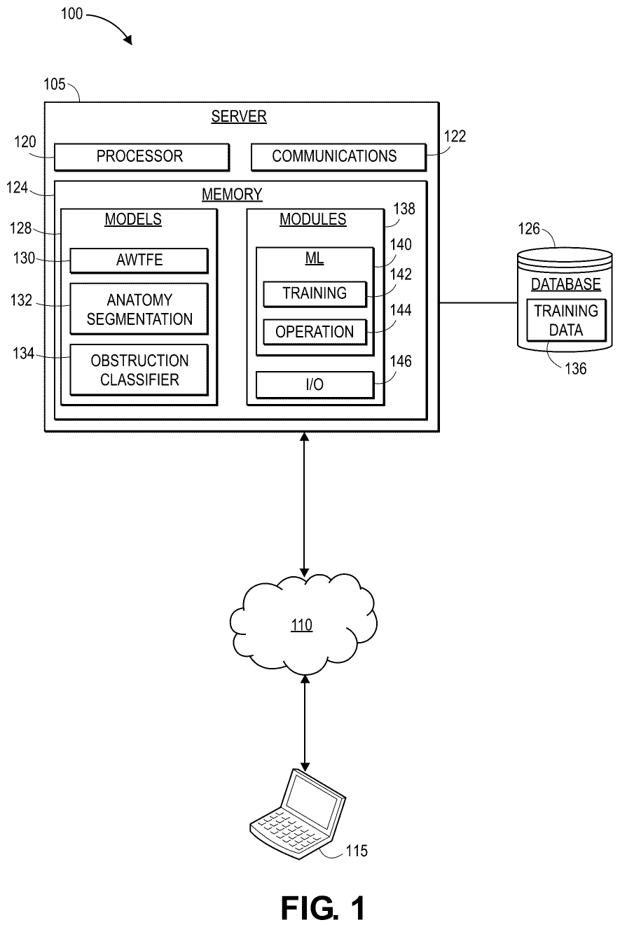

FIG. 1 depicts an exemplary computing environment 100 associated with determining and/or predicting changes in pupil morphology during an ophthalmic surgical procedure. Although FIG. 1 depicts certain entities, components, equipment, and/or devices, it should be appreciated that additional or alternate entities, components, equipment, and/or devices are envisioned.

As illustrated in FIG. 1, the computing environment 100 may include, in one aspect, at least one server 105 which may perform the at least some of the functionalities and techniques disclosed, such as determining changes in pupil morphology during an ophthalmic surgical procedure. The server 105 may be part of a cloud network or may otherwise communicate with other hardware or software components within one or more cloud computing environments to send, retrieve, or otherwise analyze data or information described herein. In one example, in certain aspects of the present techniques, the computing environment 100 may comprise an on-premise computing environment, a multi-cloud computing environment, a public cloud computing environment, a private cloud computing environment, and/or a hybrid cloud computing environment. In one example, an entity (e.g., a business providing APIs for its software applications) may host one or more services in a public cloud computing environment (e.g., Amazon Web Services (AWS), Google Cloud, IBM Cloud, Microsoft Azure, etc.). The public cloud computing environment may be a traditional off-premise cloud (i.e., not physically hosted at a location owned/controlled by the business). Alternatively, or in addition, aspects of the public cloud may be hosted on-premise at a location owned/controlled by the business. The public cloud may be partitioned using visualization and multi-tenancy techniques and/or may include one or more of software-as-a-service (SaaS), infrastructure-as-a-service (IaaS) and/or platform-as-a-service (PaaS).

The computing environment 100 may include a network 110 comprising any suitable network or networks, including a local area network (LAN), wide area network (WAN), Internet, or combination thereof. For example, the network 110 may include a wireless cellular service (e.g., 4G service, 5G service, 6G service, etc.). Generally, the network 110 enables bidirectional communication between the server 105 and a user device 115. In one aspect, the network 110 may comprise a cellular base station, such as cell tower(s), communicating to the one or more components of the computing environment 100 via wired/wireless communications based upon any one or more of various mobile phone standards, including NMT, GSM, CDMA, UMTS, LTE, 5G, 6G, or the like. Additionally or alternatively, the network 110 may comprise one or more routers, wireless switches, or other such wireless connection points communicating to the components of the computing environment 100 via wireless communications based upon any one or more of various wireless standards, including by non-limiting example, IEEE 802.11a/ac/ax/b/c/g/n (Wi-Fi), Bluetooth, and/or the like.

A communications component 122 may allow the server 105 to communicate over the network 110 via any suitable wired and/or wireless connection, e.g., using any suitable network interface controller(s) of the communications component 122. The communications component 122 may include one or more transceivers (e.g., WWAN, WLAN, and/or WPAN transceivers) functioning in accordance with IEEE reference standards, 3GPP reference standards, and/or other reference standards that may be used in receipt and transmission of data via external/network ports of the server 105 connected to computer network 110.

The server 105 may include at least one processor 120. The processor 120 may include one or more suitable processors (e.g., central processing units (CPUs) and/or graphics processing units (GPUs)). The processor 120 may be communicatively coupled to a memory 124 via a computer bus (not depicted) responsible for transmitting electronic data, data packets, or otherwise electronic signals to and from the processor 120 and memory 124 in order to execute, implement or perform the machine-readable instructions, methods, processes, elements, or limitations, as illustrated, depicted, or described for the various flowcharts, illustrations, diagrams, figures, and/or other disclosure herein. The processor 120 may interface with the memory 124 via the computer bus to execute an operating system (OS) and/or computing instructions contained therein, and/or to access other services/aspects. For example, the processor 120 may interface with the memory 124 via the computer bus to create, read, update, delete, or otherwise access or interact with the data stored in the memory 124, database 126, and/or another source of data.

The memory 124 may include one or more forms of volatile and/or nonvolatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 124 may store an operating system (OS) (e.g., Microsoft Windows, Linux, UNIX, etc.) capable of facilitating the functionalities, apps, methods, or other software as described herein. The memory 124 may store one or more sets of non-transitory, computer-executable instructions that, when executed, cause the server 105 to perform certain functions.

In general, a computer program or computer based product, application, or code (e.g., the model(s), such as ML models, or other computing instructions described herein) may be stored on a computer usable storage medium, or tangible, non-transitory computer-readable medium (e.g., reference random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 120 (e.g., working in connection with the respective operating system in memory 124) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective C, Java, Scala, ActionScript, JavaScript, HTML, CSS, XML, etc.).

The server 105 may include, or be communicatively coupled to (e.g., via network 110), at least one electronic database 126. The database 126 may include a relational database, such as Oracle, DB2, MySQL, a NoSQL database, such as MongoDB, or another electronic database. The database 126 may store, for example, surgical video data, ML model training data, ML models, and/or other suitable data.

The memory 124 and/or the database 126 may store one or more ML models 128, routines, algorithms, or other elements. The ML models 128 may be referred to as receiving an input, producing or storing an output, or executing as instructions on the processor 120. Further, the ML models 128 may be stored in the memory 124 and/or the database as executable instructions, which instructions the processor 120 may retrieve from the memory 124 and/or dataset 126 and execute. Further, the processor 120 should be understood to retrieve from the memory 124 and/or database 126 any data necessary to perform the executed instructions (e.g., data required as an input to the routine or model), and to store in the memory 124 and/or database 126 the intermediate results and/or output of any executed instructions.

In at least one aspect, the ML models 128 may include one or more ML models, as further described below. The ML models 128 may include an AWTFE ML model 130 trained to generate preprocessed image data based upon receiving surgical video data. The preprocessed image data may include one or more preprocessed images where features of the eye are extracted (e.g., extracted features associated with anatomical components of an eye), and background noise and irrelevant structures are diminished. The AWTFE ML model 130 may be trained using feature extraction training data, as further described herein. The AWTFE ML model 130 may include one or more of a two-dimensional discrete wavelet transform, higher-order tensor decomposition algorithms (e.g., Tucker algorithm), a higher-order singular value decomposition algorithm, a feature pyramid network, and/or a convolutional neural network (CNN) (e.g., VGG16 CNN). In some embodiments, adaptive wavelet tensor feature extraction of the video images may be provided by one or more algorithms (e.g., an empirically optimized algorithm) such as those just-recited rather than a trained ML model. Accordingly, in such embodiments, the systems, methods and/or techniques described herein with respect to the AWTFE ML model 130 may be executed, carried, out and/or otherwise implemented by one or more algorithms, including an optimized algorithm(s).

The ML models 128 may include an anatomy segmentation ML model 132 trained to receive surgical video data, which may include the preprocessed image data output by the AWTFE ML model 130, and generate segmented image data and/or one or more segmentation masks as an output. The segmented image data may comprise one or more segmented images wherein anatomical components of the eye are segmented. The one or more segmentation masks may be associated with the anatomical components. The anatomy segmentation ML model 132 may be trained using anatomy segmentation training data, as further described herein. The anatomy segmentation ML model 132 may include a time series transformer model and/or CNN.

The ML models 128 may include an obstruction classifier ML model 134 trained to determine the size of a pupil of the eye in the one or more segmented images. The obstruction classifier ML model 134 may be trained using obstruction classifier training data, as further described herein. The obstruction classifier ML model 134 may perform simple thresholding using distance(s) between points on the pupil and palpebral fissure boundaries. The obstruction classifier ML model 134 may include a time series transformer, a random forest and/or random sample consensus (RANSAC) algorithm. To determine the size of the pupil, the obstruction classifier ML model 134 may determine obstructed and/or unobstructed portions of the segmented images, e.g., to determine anatomical components of the eye within segmented images which are obstructed by a surgical instrument, a surgical drape, an eyelid, or any other suitable obstruction, as the obstruction may impact the accuracy of the determination of the size of the anatomical component. The determination of (un) obstructed portions of the segmented images may be made, at least in part, using one or more segmentation masks generated by the anatomy segmentation ML model 132 (e.g., by determining distances between various points of the segmentations masks, as further described below). The obstruction classifier ML model 134 may determine the size of one or more anatomical components of the eye, such as the size of the pupil and/or limbus. The determination of size may be made using unobstructed portions of the images containing the pupil and/or limbus respectively. The pupil analysis framework described herein may normalize the size of the pupil based upon the size of the limbus. For example, the pupil may change in size throughout the ophthalmic surgical procedure captured by the segmented images (e.g., due to obstructions, due to the images of the pupil being magnified by the imagining device (e.g., microscope) used to capture the images, etc.), whereas the limbus may not change in size. Accordingly, the obstruction classifier ML model 134 may use the size of the limbus (e.g., as a reference of scale) to normalize the pupil size, compensating and/or otherwise adjusting the pupil size due to effects such as magnification of the pupil, which may make the pupil appear to be larger than it actually is.

In at least some embodiments, the obstruction classifier ML model 134 may be trained to generate a prediction associated with pupil morphology (e.g., pupil size, roundness, likelihood of pupil constriction, etc.) during one or more subsequent portions of an ophthalmic surgery based upon pupil measurements and/or morphology (e.g., as indicated by morphologic data time series, spatial data, one or more the segmented images, and/or segmentation masks) in previous portions of the ophthalmic surgery (e.g., the initial stages of the of the ophthalmic surgery). In at least some embodiments, the obstruction classifier ML model 134 may generate the prediction further based upon patient medical history and/or medications (e.g., as indicated in electronic health records of the patient). The prediction of pupil morphology may be an indicator of whether a pupil expansion device may be required and/or recommended (e.g., based upon historical decisions of a surgeon as indicated by training data of one or more of the models 128) during the ophthalmic surgery. For example, the obstruction classifier ML model 134 may predict a high likelihood of pupil constriction during key phases of the ophthalmic surgery based upon the size of the pupil during one or more initial stages of the ophthalmic surgery. Based upon the prediction of pupil morphology, the server 105 may generate one or more notifications recommending the surgeon use a pupil expansion device during future portions of the ophthalmic surgery (e.g., to maintain adequate visualization throughout surgery), and provide the notification (e.g., via the network 110) to the user device 115 associated with the surgeon. Based upon the notification, the surgeon may determine whether to use the pupil expansion device.

The memory 124 and/or database 126 may store one or more sets of training data 136, such as the feature extraction training data and/or anatomy segmentation training data. The training data 136 may include testing, validation, feedback, and/or other training data which may be used to create, operate, (re) train and/or fine-tune one or more ML models 128, such as the AWTFE ML model 130, anatomy segmentation ML model 132, and/or obstruction classifier ML model 134 to generate an output (e.g., preprocessed image data, segmented image data, segmentation masks, pupil/limbus size, etc.). The training data may be, at least in part, from a public database, such as the University of Michigan BigCat database.

The memory 124 may store a plurality of computing modules 138, implemented as respective sets of computer-executable instructions (e.g., one or more source code libraries, trained ML models such as a neural network, a CNN, etc.) as described herein. For example, although depicted in FIG. 1 as part of memory 124, the AWTFE ML model 130, anatomy segmentation ML model 132 and/or obstruction classifier ML model 134 may be considered as one or more computing modules 138.

The computing modules 138 may include an ML module 140. The ML module 140 may include ML training module (MLTM) 142 and/or ML operation module (MLOM) 144. In some embodiments, at least one of the ML models 128 may be applied by the ML module 140, which may include, but are not limited to linear or logistic regression algorithms, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of ML, such as supervised learning, unsupervised learning, and reinforcement learning. In one aspect, the ML based algorithms may be included as a library or package executed on server(s) 105. For example, libraries may include the TensorFlow based library, the PyTorch library, and/or the scikit learn Python library.

In one embodiment, the ML module 140 employs supervised learning, which involves identifying patterns in existing data to make predictions about subsequently received data. Specifically, the ML module is “trained” (e.g., via MLTM 142) using training data, which includes exemplary inputs and associated exemplary outputs. Based upon the training data, the ML module 140 may generate a predictive function which maps outputs to inputs and may utilize the predictive function to generate ML outputs based upon data inputs. The exemplary inputs and exemplary outputs of the training data may include any of the data inputs or ML outputs described above. In the exemplary embodiments, a processing element may be trained by providing it with a large sample of data with known characteristics or features (e.g., the BigCat database).

In another embodiment, the ML module 140 may employ unsupervised learning, which involves finding meaningful relationships in unorganized data. Unlike supervised learning, unsupervised learning does not involve user-initiated training based upon exemplary inputs with associated outputs. Rather, in unsupervised learning, the ML module 140 may organize unlabeled data according to a relationship determined by at least one ML method/algorithm employed by the ML module 140. Unorganized data may include any combination of data inputs and/or ML outputs as described above.

In yet another embodiment, the ML module 140 may employ reinforcement learning, which involves optimizing outputs based upon feedback from a reward signal. Specifically, the ML module 140 may receive a user-defined reward signal definition, receive a data input, utilize a decision-making model to generate the ML output based upon the data input, receive a reward signal based upon the reward signal definition and the ML output, and alter the decision making model so as to receive a stronger reward signal for subsequently generated ML outputs. Other types of ML may also be employed, including deep or combined learning techniques.

The MLTM 142 may receive labeled data at an input layer of a model having a networked layer architecture (e.g., an artificial neural network, a convolutional neural network, etc.) for training the one or more ML models 128. The received data may be propagated through one or more connected deep layers of the ML model 128 to establish weights of one or more nodes, or neurons, of the respective layers. Initially, the weights may be initialized to random values, and one or more suitable activation functions may be chosen for the training process. The present techniques may include training a respective output layer of the one or more ML models 128. The output layer may be trained to output extract features associated with anatomical components of an eye, for example, based upon receiving preprocessed image data as an input.

The MLOM 144 may comprise a set of computer-executable instructions implementing ML loading, configuration, initialization and/or operation functionality. The MLOM 144 may include instructions for storing trained ML models (e.g., as the ML models 128 in the memory 124). As described, once trained, the one or more trained ML models 128 may be operated in inference mode, whereupon when provided with de novo input that the model has not previously been provided, the model may output one or more predictions, classifications, etc., as described herein.

In operation, the MLTM 142 may access memory 124, database 126, and/or any other data source for training data (e.g., training data 136) suitable to generate one or more ML models, such as ML models 128. The training data may be sample data with assigned relevant and comprehensive labels (classes or tags) used to fit the parameters (weights) of the ML model 128 with the goal of training it by example. In one aspect, once an appropriate ML model 128 is trained and validated to provide accurate predictions and/or responses, the trained ML model 128 may be loaded into MLOM 144 at runtime to process input data and generate output data.

While various embodiments, examples, and/or aspects disclosed herein may include training and generating one or more ML models 128 for the server 105 to load at runtime, one or more appropriately trained ML models may already exist (e.g., ML models 128 in memory 124) such that the server 105 may load an existing trained ML model 128 at runtime. The server 105 may retrain, finetune, update and/or otherwise alter an existing ML model 128 before and/or after loading the model 128 at runtime. Although the ML model 128 is described as being trained (e.g., via MLTM 142) and operated (e.g., via MLOM 144) on the server 105, in at least one embodiment the ML model 128 may be trained one server 105, and operated on another server 105.

In one aspect, the computing modules 138 may include an input/output (I/O) module 146, comprising a set of computer executable instructions implementing communication functions. The I/O module 146 may include a communication component configured to communicate (e.g., send and receive) data via one or more external/network port(s) to one or more networks or local terminals, such as the computer network 110 described herein. In one aspect, the server 105 may include a client-server platform technology such as ASP.NET, Java J2EE, Ruby on Rails, Node.js, a web service or online API, responsive for receiving and responding to electronic requests.

The I/O module 146 may further include or implement a user interface configured to present information to an administrator, operator or other user, and/or receive inputs from the user, such as via a touchscreen display. The I/O module 146 may facilitate I/O components (e.g., ports, capacitive or resistive touch sensitive input panels, keys, buttons, lights, LEDs), which may be directly accessible via, or attached to, the server 105 and/or may be indirectly accessible via, or attached to, another device. According to one aspect, user may access the server 105 via a user interface to review information, make changes, input training data, initiate training via the MLTM 142, and/or perform other functions (e.g., operation of one or more trained models via the MLOM 144).

The server 105 may also be in communication with one or more user devices 115, e.g., a user device associated with a user requesting information/data from, and/or providing information/data to, the server 105, such the AWTFE ML model 130, anatomy segmentation ML model 132, obstruction classifier ML model 134, etc. The user device 115 may comprise one or more computers and/or multiple, redundant, or replicated client computers accessed by one or more users. The user device 115 may access services and/or other components of the computing environment 100 via the network 110, as further described below. The user device 115 may include one or more computing devices (e.g., desktop computer, laptop computer, terminal), mobile devices, wearables, medical devices (surgical system and/or display), surgical tools, augmented reality glasses/headsets, virtual reality glasses/headsets, mixed or extended reality glasses/headsets, and/or other suitable electronic or electrical components. The user device 115 may include a memory and a processor for, respectively, storing and executing one or more modules, computer-executable instructions, etc. The memory may include one or more suitable storage media such as a magnetic storage device, a solid-state drive, random access memory (RAM), etc.

In some embodiments, the computing environment 100, e.g., via server 105, may determine and/or predict changes in pupil morphology during an ophthalmic surgical procedure, such as a cataract surgery, a vitreoretinal surgery, or a corneal surgery. The server 105 may receive surgical video data comprising one or more images (e.g., color images) of at least a portion of the eye during at the ophthalmic surgical procedure. For example, the user device 115 such as a camera, imaging device, and/or surgical equipment, may generate and provide the surgical video data associated with an entire cataract surgery to the server 105, e.g., via an electronic communication over network 110, a storage device of the user device 115 communicatively coupled to the server 105 via I/O module 146, and/or in any other suitable manner. The server 105 may store the surgical video data in the memory 124 and/or the database 126, provide the surgical video data to one or more ML models 128, etc.

The server 105 may provide the surgical video data to the AWTFE ML model 130 to generate preprocessed image data comprising one or more preprocessed images. The preprocessed images may include extracted features associated with anatomical components of the eye, such as the pupil, limbus, sclera, palpebral fissure, or other suitable anatomical component. The preprocessed images may eliminate noise from the surgical video data images, extract and/or enhance distinctive features of the pupil, limbus, palpebral fissure, and/or other anatomical components within the image (e.g., by extracted features to emphasize object boundaries, textures, shapes, and/or other distinctive attributes), among other things. The preprocessing image data may improve the performance of the anatomy segmentation ML model 132 when segmenting the preprocessed images of the preprocessed image data. The server 105 may store the preprocessed image data in the memory 124 and/or the database 126, e.g., to use for ML model training.

In at least one aspect, the AWTFE ML model 130 may generate a third-order tensor from one or more color images of the surgical video data and/or preprocessed images which may represent the correlations among spatial information, color channels, and wavelet subbands of the color image(s) taken during surgery. Specifically, a third-order tensor of a color pupil image may be constructed using twelve wavelet subbands of three color channels (e.g., red, green, blue channels) to represent the correlations in a higher data dimension.

The server 105 may provide the preprocessed image data to the anatomy segmentation ML model 132 as an input. In at least some embodiments, the anatomy segmentation ML model 132 may receive the surgical video data as the input which is not preprocessed by the AWTFE ML model 130. The anatomy segmentation ML model 132 may generate segmented image data as an output. The segmented image data may include segmented image(s) wherein the anatomical components of the eye are segmented, such as the pupil, limbus, sclera, palpebral fissure, and/or any other suitable anatomical component of the eye. For example, the segmented image data may include one or more images of a segmented pupil.

Using the disclosed techniques, one or more of the models ML models 128 (e.g., the anatomy segmentation ML model 132) and/or the server 105 (e.g., via the processor 120) may generate a semantic segmentation of the pupil in real-time for one or more frame of the surgical video. The disclosed techniques may include generating morphologic data time series using the one or more images of the semantically segmented pupil. The morphologic data time series may include a sequence of data points collected over time that describe the shape, structure, or form of an object, such as the pupil or other morphology. For example, one or more of the models ML models 128 and/or the server 105 may use one or more images of the semantically segmented pupil to compute a time series of one or more parameters of a connected region of the eye and/or pupil, such as circularity, major/minor axis lengths, area, convex hull, convex area, eccentricity, perimeter length, and/or centroid location. Moreover, binary characteristics, also referred to as “spatial data,” may be computed one or more time points of the surgical procedure associated with the surgical video, such as iris tissue entrapment in incisions, whether a pupil expansion device is present, etc. In at least some embodiments, the disclosed techniques may compute the binary characteristics over short time periods (e.g., 1-2 seconds), binary characteristics such as iris billowing (rapid fluctuation in pupil size) and/or pupillary constriction.

The anatomy segmentation ML model 132 may generate from the preprocessed image data one or more segmentation masks associated with the anatomical components. Segmenting the anatomical components in the segmented image data, and generating segmentation masks associated with the anatomical components, may improve detection and compensation of obstructions (e.g., by the obstruction classifier ML model 134) in the images, among other things. The server 105 may similarly store the segmented image data and/or segmentation masks in the memory 124 and/or the database 126, e.g., to use for ML model training.

The server 105 may determine via the obstruction classifier ML model 134 a size of a pupil of the eye in the one or more segmented images and/or predict pupil morphology, (e.g., during one or more portions of the ophthalmic surgery). The determination may include determining one or more obstructed portions of the one or more segmented images (e.g., obstructions from a surgical instrument, an eyelid, a surgical drape, etc.) using the one or more segmentation mask. For example, the obstruction classifier ML model 134 may determine distances and/or other metrics between one or more portions (e.g., edges, contours, etc.) of one or more segmentation masks (e.g., the pupil segmentation mask and limbus segmentation mask) to determine whether there is an obstruction (e.g., an obstruction of the anatomical component associated with the segmentation masks used in the distance measurements). This may include comparing distances and/or metrics to certain thresholds which may indicate an obstruction, as further described herein.

The obstruction classifier ML model 134 may determine and/or predict the size of the pupil or other pupil morphology, e.g., using one or more segmented images of the pupil (e.g., associated with unobstructed portions of the pupil, morphologic data time series, spatial data, etc.). This may include analyzing characteristics of the pupil (e.g., size, edges, contours, areas of portions of the pupil, distance to other anatomical components, etc.), the pupil mask (e.g., the size of the pupil segmentation mask if the pupil is unobstructed), among other things, in one or more of the segmented images to determine the size of the pupil. The size of the pupil may include determining other metrics associated with the eye and/or pupil, such as area, eccentricity, convex hull, major axis length, minor axis length, contour path length, etc. At least some of these metrics may be used to detect the shape of the pupil, which if not round, may be indicative of eye-related issues, such intraoperative floppy iris syndrome (IFIS) (e.g., indicated by the iris entrapped in a wound using the metrics and/or shape anatomical components), the presence of vitreous in the anterior chamber (e.g., when detecting a peaked pupil using the metrics and/or shape anatomical components), among other things. The prediction of pupil morphology may include, and/or indicate, a predicted size and/or roundness of the pupil for one or more subsequent phases of the ophthalmic surgical procedure, the need and/or benefit of a pupil expansion device during one or more subsequent phases of the ophthalmic surgical procedure, development of iris billowing, development of entrapment of iris in incisions, and/or any other suitable prediction. For example, the predicted size of the pupil may be an indicator of whether a pupil expansion device may be required and/or recommended during one or more stages of the surgery

In at least one aspect, the images of the eye/pupil may be magnified, e.g., based upon the level of magnification when recording the surgical video. The level of magnification may stay the same or change throughout the surgical video, e.g., due to preferred level of magnification of the user recording the surgical video, which may cause the pupil to appear to be a different size than it actually is, e.g., changing in size in the surgical images simply due to magnification of the pupil in the images. Thus, to determine whether the pupil is actually changing in size throughout the surgical procedure (e.g., due to dilation of the pupil) versus whether the pupil is appearing to change in size (e.g., due to magnification of the pupil/surgical images), or some combination thereof, the obstruction classifier ML model 134 may normalize to the size of the pupil to determine its actual size.

To address this challenge, the pupil size which was determined by the obstruction classifier ML model 134 may be normalized using the corresponding size of the limbus region. In contrast to the pupil which may change in size (e.g., due to dilation, magnification, etc.), the size of the limbus region may be physically fixed and remain significantly unaltered under normal circumstances during one or more phases of the surgical procedure (e.g., phacoemulsification). This allows the size of the limbus to be used as a reference for determining the size of the pupil and compensate for effects due to magnification of the pupil rather than an actual change in size of the pupil. However, similar to the pupil, the apparent size of the limbus in the surgical video images may be affected by obstructions which may obscure the limbus. Therefore, to ensure the accuracy of the server 105 when determining the size the limbus, which will in turn be used to normalize the size of the pupil, the obstruction classifier ML model 134 may determine the size of the limbus of the eye using unobstructed portions of the limbus in the one or more segmented images. This may include a similar process as that just described with respect to the pupil, such as determining the size of the limbus and the pupil using the corresponding segmented images and/or during the same phase of the surgical procedure. The obstruction classifier ML model 134 may then normalize the size of the pupil using the size of the limbus.

The server 105 may determine the size of the pupil of the eye in the one or more segmented images associated with a multiple phases of the ophthalmic surgical procedure. In at least one aspect, this may include the process just described with respect to determining the pupil size for a first phase of surgery, and then determining the size again for a second phase of surgery, i.e., at least (i) receiving surgical video data associated with the second phase of the surgery (if not already included in the surgical video of the first phase)(ii) providing the surgical video data associated with the second phase of the ophthalmic surgical procedure to the AWTFE ML model 130 to generate preprocessed image data associate with the second phase; (iii) providing the second phase preprocessed image data to the anatomy segmentation ML model 132 to generate segmented image data and segmentations masks associated with the second phase; and (iv) determining the size of the pupil during the second phase. In some aspects, determining the size of the pupil during the second phase may include fewer steps than just described. In one example, if the surgical video data first received when determining pupil size during the first phase of surgery also includes the second phase of surgery, the server 105 may determine the size of the pupil during the second phase based upon the first-received surgical video data.

In one example, when the surgical video data includes the first and second phases of surgery, the server 105 may determine the size of the pupil of the eye in the one or more segmented images associated with the second phase of the ophthalmic surgical procedure as just described with respect to the first portion of the surgery. The server 105 may determine a change in the size of the pupil during at least a portion of the ophthalmic surgical procedure associated with the first and second phases of the ophthalmic surgical procedure based upon comparing the size of the pupil during the first and second phases of the ophthalmic surgical procedure. For example, if the size of the pupil is 80 millimeters (mm) during the main wound (second) phase of a cataract surgery and 60 mm during the medication injection (first) phase of the cataract surgery, the comparison indicates the pupil has increased in size by 20 mm, e.g., due to dilation of the pupil from the medication injection.

In at least one aspect, the server 105 may determine the size of the pupil of the eye in one or more segmented images associated with more than two phases of the ophthalmic surgical procedure (e.g., all phases of the ophthalmic surgical procedure). In such an aspect, determining the change in the size of the pupil may be further based upon comparing the size of the pupil during multiple phases of the ophthalmic surgical procedure (e.g., comparing the size of the pupil through every phase of a cataract surgery described above).

The server 105 may generate one or more notifications, e.g., at one or more times during the ophthalmic surgery. The notification may be associated with the one or more predictions and/or changes in the size of the pupil, the shape of the pupil, and/or other pupil metrics or morphologies (e.g., eccentricity, convex hull, major axis length, minor axis length, contour path length, etc.) as determined at one or more times during an ophthalmic surgery. For example, the notification may indicate the actual and/or predicted size and/or roundness of the pupil during one or more phases of surgery, the actual and/or predicted change in size of the pupil during various phases of surgery, a recommendation of a pupil expansion device (e.g., to use a pupil expansion device during one or more phases of surgery), development of iris billowing, development of entrapment of iris in incisions, a billing code associated with the ophthalmic surgical procedure (e.g., a complex or non-complex surgery billing code), a prediction associated with intraoperative floppy iris syndrome IFIS (e.g., that the subject of the surgery has IFIS, will develop IFIS, is part of a drug trial to determine whether the drug causes IFIS, etc.), and/or any other suitable notification.

The server 105 may provide the notification to the user device 115. The notification may be a visual notification, such as a text message, a symbol, a graphic, a warning; an audio notification such as a buzzer, verbal notification (e.g., provided by a chatbot); and/or any other suitable notification. The notification may be displayed and/or output on a user device 115 such as a surgical system display, a desktop computer, a laptop, a mobile device, a head-mounted surgical display, speakers, and/or any other suitable user device 115.

Although the computing environment 100 is shown to include one each of the server 105, the network 110, and the user device 115, it should be understood that different numbers of servers 105, networks 110 and/or user devices 115 may be utilized. In one example, the computing environment 100 may include hundreds of servers 105 all of which may be interconnected via the network 110 to dozens of user devices 115.

The computing environment 100 may include additional, fewer, and/or alternate components, and may be configured to perform additional, fewer, or alternate actions, including components/actions described herein. For example, although the server 105 is shown in FIG. 1 as including one instance of various components such as the processor 120, the memory 124 and the database 126, various aspects include the computing environment 100 and/or the server 105 implementing any suitable number of any of the components shown in FIG. 1 and/or omitting any suitable ones of the components shown in FIG. 1. For instance, information described as being stored in the memory 124 may be stored in the database 126, and therefore the memory 124 may be omitted. Moreover, various aspects include the computing environment 100 having any suitable number of additional component(s) not shown in FIG. 1, such as but not limited to the exemplary components described above. Furthermore, it should be appreciated that additional and/or alternative connections between components shown in FIG. 1 may be implemented. As just one example, server 105 may be connected to database 126 via network 110 rather than appearing to be connected via a direct connection as illustrated in FIG. 1.

### Exemplary User Device

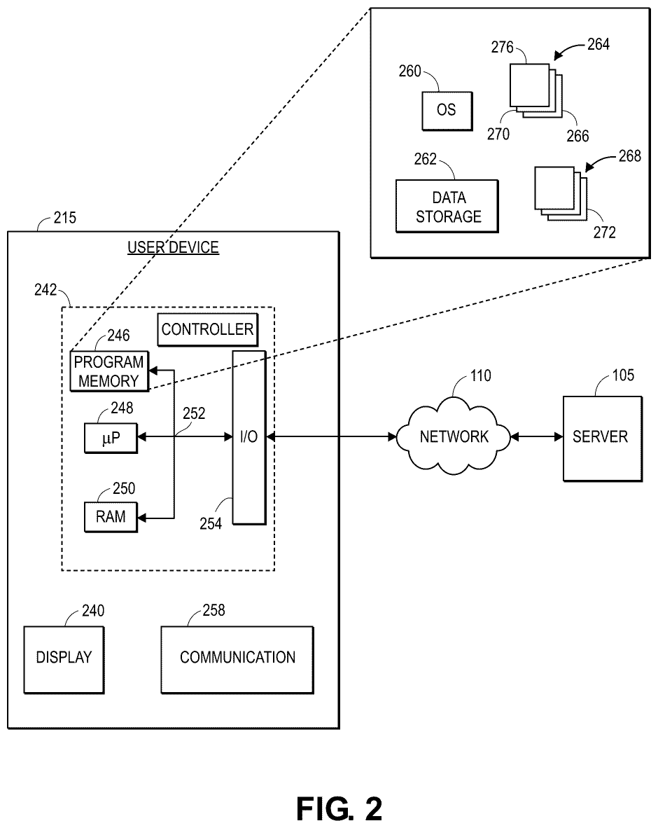

FIG. 2 depicts an exemplary user device 215, such as the user device 115, for determining pupil morphology during an ophthalmic surgical procedure. The user device 215 may include a display 240, a communication component 258, a user-input device (not shown), and a controller 242. The controller 242 may include a program memory 246, a microcontroller/processor/microprocessor (μP) 248 such as the processor 120, a random-access memory (RAM) 250, and/or an input/output (I/O) circuit 254, all of which may be interconnected via an address/data bus 252. The program memory 246 may include an operating system 260, a data storage 262, a plurality of software applications 264, and/or a plurality of software routines 268. The operating system 260, for example, may include one of a plurality of platforms such as the MacOS or iOS by Apple, Windows or Windows Mobile by Microsoft, Unix, ChromeOS or Android by Google, etc.

The data storage 262 may include data such as application data for the plurality of applications 264, routine data for the plurality of routines 268, and/or other data necessary to interact with the one or more servers 105 through the network 110. In some embodiments, the controller 242 may also include, or otherwise be communicatively connected to, other data storage mechanisms (e.g., one or more hard disk drives, optical storage drives, solid state storage devices, etc.) that reside within the user device 215.

The communication component 258 may communicate with the one or more components or devices, such as server 105, via any suitable wireless communication protocol network, such as a wireless telephony network (e.g., GSM, CDMA, LTE, 5G, 6G, ultrawideband, etc.), a Wi-Fi network (e.g., having 802.11 standards), a WiMAX network, a Bluetooth network, etc. The user-input device (not shown) may include a “soft” keyboard that is displayed on the display 240 of the user device 215, an external hardware keyboard communicating via a wired and/or a wireless connection (e.g., a Bluetooth keyboard), an external mouse, a touchscreen, a stylus, and/or any other suitable user-input device.

The one or more processors 248 may be adapted and/or configured to execute any one or more of the plurality of software applications 264 and/or any one or more of the plurality of software routines 268 residing in the program memory 246, in addition to other software applications.

One of the plurality of applications 264 may be a native application and/or web browser 270, such as Apple's Safari, Google's Chrome, Microsoft's Edge, Mozilla's Firefox, that may be implemented as a series of machine-readable instructions for receiving, interpreting, and/or displaying application screens or web page information from the one or more servers 105 while also receiving inputs from the user. Another application of the plurality of applications may include an embedded web browser 276 that may be implemented as a series of machine-readable instructions for receiving, interpreting, and/or displaying web page information.

One of the plurality of applications 264 may be an application for performing the various tasks and/or functions associated with determining changes in pupil morphology during an ophthalmic surgical procedure, such as displaying a user interface (UI) for receiving notifications associated with the change in the size of the pupil, displaying information on, and/or transmitting information from, the user device 215 to the server 105, etc. Additionally, the user may also launch or instantiate any other suitable user interface application (e.g., the native application or web browser 270, and/or any other one of the plurality of software applications 264) to access the one or more servers 105 to realize aspects of the inventive system.

The user device 215 may include additional, fewer, and/or alternate components, and may be configured to perform additional, fewer, or alternate actions, including components/actions described herein. Although the user device 215 is shown in FIG. 2 as including one instance of various components such as display 240, processor 248, etc., various aspects include the user device 215 implementing any suitable number of any of the components shown in FIG. 2 and/or omitting any suitable ones of the components shown in FIG. 2. Moreover, various aspects include the user device 215 including any suitable additional component(s) not shown in FIG. 2, such as but not limited to the exemplary components described with respect to other figures, such as FIG. 1. Furthermore, it should be appreciated that additional and/or alternative connections between components shown in FIG. 2 may be implemented.

### Exemplary ML Model Training

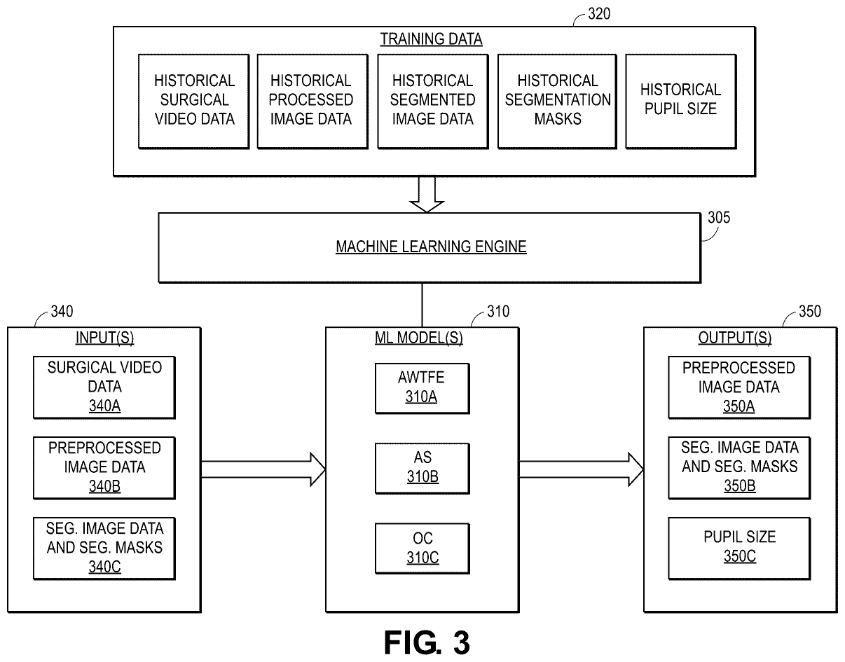

FIG. 3 schematically illustrates how an ML model 310 may be trained to generate an output 350 based upon receiving an input 340, such as training the ML models 310A-310C to determine pupil morphology during an ophthalmic surgical procedure. The ML model 310 may be and/or include AWTFE ML model 310A such as AWTFE ML model 130, anatomy segmentation ML model 310B such as anatomy segmentation ML model 132, and/or obstruction classifier ML model 310C such as obstruction classifier ML model 134. Some of the blocks in FIG. 3 may represent hardware and/or software components (e.g., ML engine 305), other blocks may represent data structures or memory storing these data structures, registers, or state variables (e.g., training data 320), and other blocks represent output data (e.g., output 350).

The ML engine 305 may include one or more hardware and/or software components (e.g., the ML module 140, the MLTM 142 and/or the MLOM 144) to obtain, create, (re) train, operate, fine-tune, and/or store one or more ML models 310. To train the ML model 310, the ML engine 305 may use training data 320. A server such as server 105 may obtain and/or have available one or more types of training data 320 (e.g., training data stored in database 126). In one aspect, at least some of the training data 320 may be labeled to aid in (re) training and/or fine-tuning the ML model 310. The ML model 310 may be configured to process the training data 320 to learn associations and relationships in the training data 320. The ML engine 305 may process and/or analyze the training data 320 (e.g., via MLTM 142) to train the ML model 310 to generate the output 350. The ML model 310 may be trained via regression, k-nearest neighbor, support vector regression, and/or random forest algorithms and/or models, although any type of applicable ML algorithm and/or model may be used, including training using one or more of supervised learning, unsupervised learning, semi-supervised learning, and/or reinforcement learning.

In one aspect, the AWTFE ML model 310A may be trained to receive surgical video data 340A as an input 340 and generate preprocessed image data 350A as an output 350. The training data 320 for the AWTFE ML model 310A, at times referred to herein as feature extraction training data, may include historical surgical video data from historical ophthalmic surgical procedures, historical processed image data having extracted features associated anatomical components an eye, or any other suitable training data 320. The AWTFE ML model 310A may be trained to learn associations and relationships in such training data 320. In one example, the training data 320 for the AWTFE ML model 310A may include an historical surgical video of an historical cataract surgery and may include labels indicating the anatomical components of the eye in the historical surgical video, such as the pupil, the limbus, and the sclera. Historical processed image data may include labels corresponding to extracted features associated with the pupil, the limbus, and the sclera. The historical surgical video and historical processed image data may be provided as training data 320 to the AWTFE ML model 310A to train the AWTFE ML model 310A to make associations between the pupil in the historical surgical video and the corresponding extracted feature of the pupil in the historical processed image data, the limbus in the historical surgical video and the corresponding extracted feature of the limbus in the historical processed image data, the sclera in the historical surgical video and the corresponding extracted feature of the sclera in the historical processed image data, and/or any other suitable associations, such as associations of other anatomical components of the eye in the historical surgical video and the historical processed image data. In at least one aspect, the AWTFE ML model 310A may be considered as successfully trained once it is able to achieve one or more metrics (e.g., precision score, recall score, etc.) associated with its performance when processing the training data 320, e.g., its performance when generating preprocessed image data from surgical video data.

In one aspect, the anatomy segmentation ML model 310B may be trained to receive as an input 340 surgical video data, such as the preprocessed image data 340B (e.g., the preprocessed image data 350A output by the AWTFE ML model 310A) and generate segmented image data and/or segmented image masks 350B as an output 350. The segmented image data may include morphologic data time series and/or spatial data, as previously described. The training data 320 for the anatomy segmentation ML model 310B, at times referred to herein as anatomy segmentation training data, may include historical surgical video data, historical preprocessed image data having extracted features associated with historical anatomical components of eyes, historical segmented image data segmenting the historical anatomical components, historical segmented image masks of the historical anatomical components, historical morphologic data time series, historical spatial data, and/or any other suitable training data 320. The anatomy segmentation ML model 310B may be trained to learn associations and relationships in such training data 320. In one example, the training data 320 for the anatomy segmentation ML model 310B may include historical processed image data including labels corresponding to extracted features associated with historical anatomical components of the eye, such as the pupil and the limbus, historical segmented image data including labels corresponding to the associated segmented historical anatomical components, such as the pupil and limbus, and/or historical segmented image masks including labels corresponding to the associated historical anatomical components, such as the pupil and limbus. The historical processed image data, historical segmented image data, and/or historical segmented image masks may be provided as training data 320 to the anatomy segmentation ML model 310B to train the anatomy segmentation ML model 310B to make associations, such as associations between the extracted features of the pupil and limbus in the historical preprocessed image data, the corresponding segmented pupil and limbus in the historical segmented image data, and the corresponding pupil and limbus segmentation masks in the historical segmentation masks. In at least one aspect, the anatomy segmentation ML model 310B may be considered as successfully trained once it is able to achieve one or more metrics (e.g., precision score, recall score, etc.) associated with its performance when processing the training data 320, e.g., its performance when generating segmented images and segmentation masks from associated preprocessed image data.

In one aspect, the obstruction classifier ML model 310C may be trained to receive segmented image data (e.g., morphologic data time series and spatial data) and/or segmented image masks 340C as an input 340 and determine and/or predict the size of a pupil 350C and/or other pupil morphology as an output 350. The training data 320 for the obstruction classifier ML model 310C, at times referred to herein as obstruction classifier training data, may include historical segmented image data having segmented historical anatomical components of historical eyes, historical segmented image masks of the historical anatomical components, historical morphologic data time series, historical spatial data, historical pupil sizes of historical pupils, historical pupil morphology predictions of historical pupils, historical patient medical record information (e.g., medical history and/or medications), and/or other suitable training data 320. The obstruction classifier ML model 310C may be trained to learn associations and relationships in such training data 320. In one example, the training data 320 for the obstruction classifier ML model 310C may historical segmented image data including labels corresponding to a segmented pupil and limbus, historical segmented image masks including labels corresponding to the associated segmented pupil and limbus, and an associated historical pupil size which may include a corresponding label, and/or other historical anatomical components. The historical segmented image data, historical segmented image masks, and historical pupil size may be provided as training data 320 to the obstruction classifier ML model 310C to train the obstruction classifier ML model 310C to make associations between the segmented features of the pupil and limbus (or other anatomical components), in the historical segmented image data, corresponding pupil and limbus (or other anatomical components) segmentation masks in the historical segmentation masks, and corresponding historical pupil and/or limbus size (or other anatomical components). In at least one aspect, the obstruction classifier ML model 310C may be considered as successfully trained once it is able to achieve one or more metrics (e.g., precision score, recall score, etc.) associated with its performance when processing the training data 320, e.g., its performance when generating a pupil size from segmented images and segmentation masks of an associated pupil and limbus.

The server and/or ML engine 305 may continuously update the training data 320. For example, when a trained ML model 310 generates an output data 350 based upon input data 340, the ML engine 305 may store the input data 340 and/or output data 350 as updated training data 320. Subsequently, the ML model 310 may be retrained based upon the updated training data 320, which cause the output 350 of the ML model 310 to improve over time. For example, when providing a new surgical video 340A to AFTWE 310A to generate preprocessed image data 350A, subsequently providing the preprocessed image data 340B (previously generated as 350A by AWTFE ML model 310A) to the anatomy segmentation ML model 310B to generate the segmentation image data and segmentation masks 350B, and providing the segmentation image data and segmentation masks 340C (previously generated as 350B by anatomy segmentation ML model 310B) to the obstruction classifier ML model 310C to generate the pupil size 350C, any of the inputs 340A-340C and/or outputs 350A-350C of the ML models 310A-310C may be stored as updated training data 320.

Once trained, the ML model 310 may perform operations on one or more data inputs 340 to produce the desired data output 350. In one aspect, the ML model 310 may be loaded at runtime from a database (e.g., by ML engine 305 from the database 126) to process the input data 340. The server and/or ML engine 305 may obtain the input data 340 (e.g., from the database 126), and provide the input data 340 to the trained ML model 310 to generate the output 350.

For example, a new surgical video 340A of an entire cataract surgery of Patient A may be provided to AWTFE ML model 310A to generate preprocessed image data 350A for all phases of the surgery. The preprocessed image data 350A may include extracted features of the pupil, limbus, sclera, and palpebral fissure. The preprocessed image data 350A may then be provided as an input 340 to the anatomy segmentation ML model 310B to generate segmented image data and segmentations masks 350B for anatomical components of the eye of Patient A through all phases of the surgery, such as segmented images and masks of the pupil, limbus, sclera, and palpebral fissure. The segmented image data and segmentations masks 350B may be provided as inputs 340C to the obstruction classifier ML model 310C to determine and/or predict the size of Patient A's pupil 350C during one or more phases of the surgery, which may include determining the size of the limbus to normalize the size of the pupil and/or predicting pupil morphology indicating that a pupil expansion device may be required and/or recommended during the surgery. The server may generate one or more notifications based upon a comparison of the pupil size of Patient A during the different phases of the cataract surgery and/or the prediction of Patient A's pupil morphology during one or more phases of the cataract surgery. The notification may indicate that Patient A's is experiencing IFIS and/or the predicted pupil morphology indicates a likelihood of pupil constriction during key phases of surgery. The notification may provide a recommendation to the surgeon to use a pupil expansion device during at least a portion of the cataract surgery based upon the IFIS and/or predicated pupil morphology of Patient A.

### Exemplary High-Level System Flow for Determining Pupil Morphology During an Ophthalmic Surgical Procedure

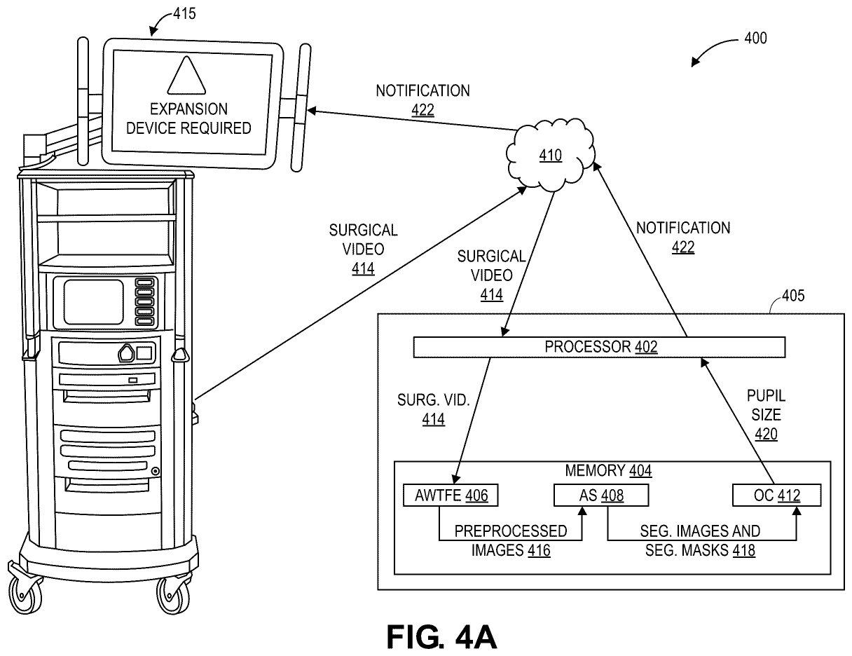

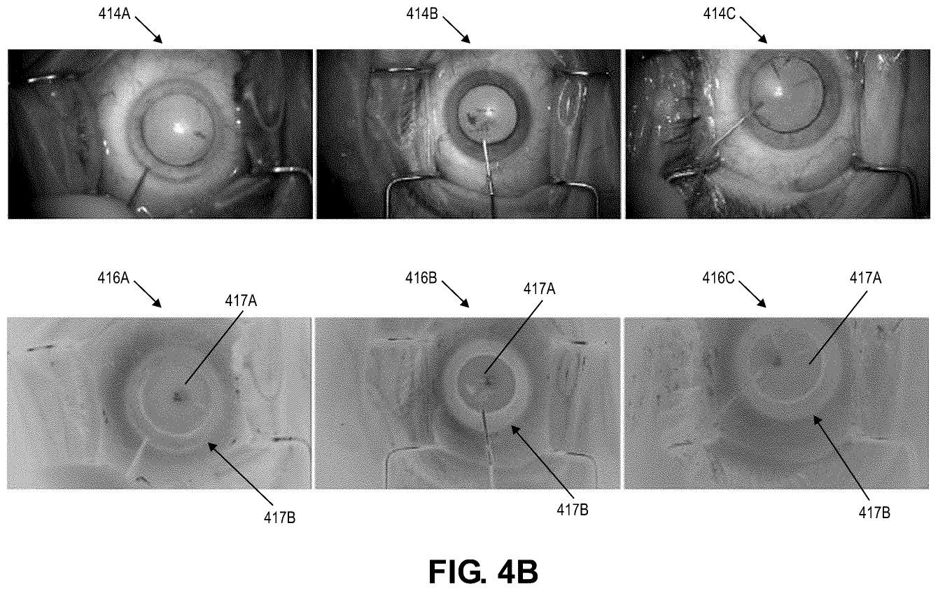

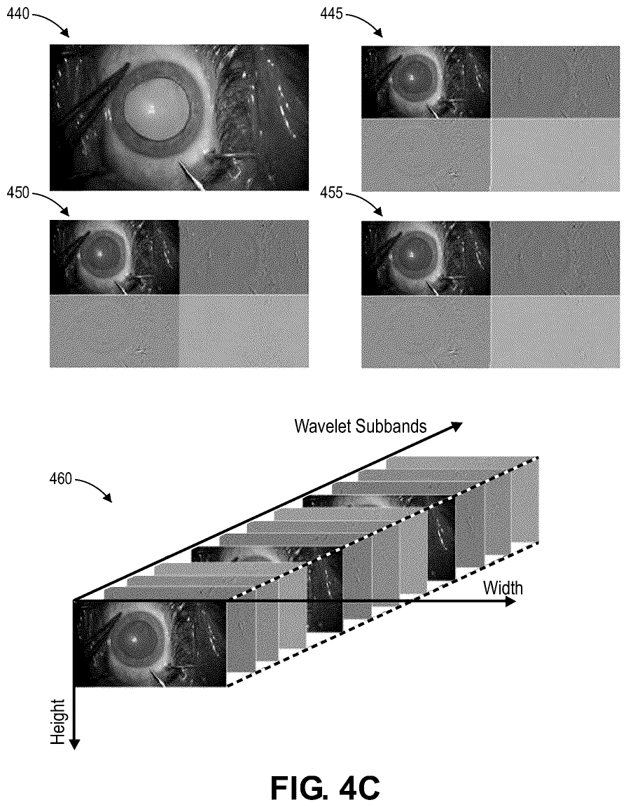

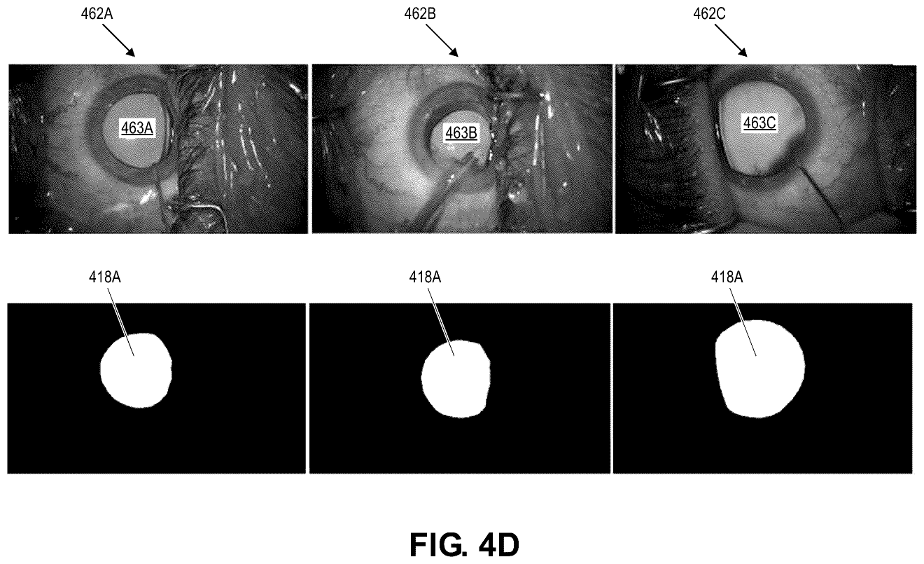

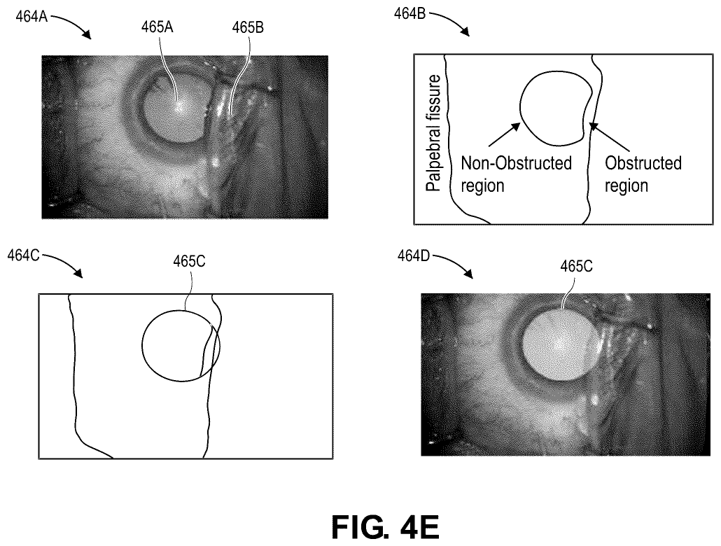

FIG. 4A is an exemplary block diagram depicting a high-level system flow for determining pupil morphology during an ophthalmic surgical procedure. In general, the system flow may be carried out by devices, models, and/or components of a Pupil Morphology System 400, such the devices, models, and/or components of the computing environment 100.

The Pupil Morphology System 400 may include at least one server 405 (e.g., server 105) communicatively connected via a network 410 (e.g., network 110) to a surgical system 415 (e.g., user device 115, 215). The server 405 may include a processor 402 such as processor 120, a memory 404 such as memory 124, an AWTFE ML model 406 such as AWTFE ML models 130, 310A, an anatomy segmentation ML model 408 such as anatomy segmentation ML models 132, 310B, and an obstruction classifier ML model 412 such as obstruction classifier ML models 134, 310C.

According to an example embodiment of the Pupil Morphology System 400, a surgeon may be conducting a cataract surgery using the surgical system 415. The surgical system 415 may provide data including surgical video images 414 (e.g., surgical video data) of the entire surgery in real-time to the server 405 via the network 410. The processor 402 of the server 405 may receive the surgical video images 414 via a communications module (not shown) such as communications component 122, and further provide the surgical video images 414 to the AWTFE ML model 406 stored in memory 404.

The AWTFE ML model 406 may process the surgical video images 414, e.g., in real-time, to generate preprocessed image data 416. This may include extracting features of anatomical components of the eye of the patient from one or more images of the surgical video images 414 to generate the preprocessed image data 416. FIG. 4B illustrates three exemplary ophthalmic surgical procedure video images 414A-414C of the surgical video images 414 of the patient's eye during different phases of the surgery, and three exemplary corresponding feature-extracted images 416A-416C from the preprocessed image data 416. In the preprocessed images 416A-416C, the AWTFE ML model 406 has extracted features associated with the pupil 417A and the limbus 417B.

In at least one aspect, to generate the preprocessed image data 416, the AWTFE ML model 406 may be trained to generate a third-order tensor of the pupil based upon a color image of the pupil in the surgical video images 414. FIG. 4C illustrates an exemplary ophthalmic surgical procedure color image 440, red channel wavelets images 445, green channel wavelets images 450, blue channel wavelets images 455, and an exemplary third-order wavelet subband tensor (tensor) 460. The third-order wavelet subband tensor 460 of the pupil may be based upon the color image 440. The third-order wavelet subband tensor 460 is designed to model the correlations among various data dimensions in the color pupil image 440. The third-order wavelet subband tensor 460 construction may use wavelet subbands of the different frequency components of the color image 440 that are decomposed using wavelets. A given image can be decomposed into several multiresolution frequency components called subbands which contain changes of the image along with vertical, horizontal, and diagonal directions. The third-order wavelet subband tensor 460 construction using wavelet subbands involves stacking the wavelet coefficients of each subband together to form a multi-dimensional tensor, where each dimension represents a different subband at a different scale. According to the present systems, methods and techniques, the third-order wavelet subband tensor 460 construction represents the correlations among data dimensions of the color pupil image 440, more specifically using twelve subbands of the color pupil image 440, which has been shown to effectively capture correlations among color channels, spatial information, and wavelet subbands. Given the color pupil image 440, the third-order wavelet subband tensor 460 may be generated from wavelet subbands of a red channel as illustrated by images 445, wavelet subbands of a green channel as illustrated by images 450, and wavelet subbands of a blue channel as illustrated by images 455. The color channels, when isolated, provide intensity-only images (e.g., intensity of the associated color channel), such that the color channel images 445, 450, 455 may render as grayscale images indicative of the intensity, as illustrated in FIG. 4C.

Returning to FIG. 4A, the server 405 may provide (e.g., via processor 402) the preprocessed image data 416 generated by the AWTFE ML model 406 as an input to the anatomy segmentation ML model 408. The anatomy segmentation ML model 408 may generate segmented images 417 of the anatomical components of the eye in the preprocessed images of the preprocessed image data 416, such as anatomical components (e.g., pupil, limbus, etc.) which have been extracted from the surgical video images 414 by the AWTFE ML model 406. The anatomy segmentation ML model 408 may generate segmentation masks 418 associated with the segmented anatomical components of the segmented images 417. In at least one aspect, the anatomy segmentation ML model 408 may include a CNN to generate the segmented images 417 and/or segmentations masks 418. Turning to FIG. 4D, three exemplary images 462A-462C of pupils 463A-463C during a surgical procedure are illustrated, as well as segmentation masks 418A-418C associated with the pupils 463A-463C of images 462A-462C.

Returning again to FIG. 4A, the server 405 may provide (e.g., via processor 402) the segmented images 417 and segmentation masks 418 generated by the anatomy segmentation ML model 408 to the obstruction classifier ML model 412. The obstruction classifier ML model 412 may generate the size of the pupil 420 in one or more phases of the surgery associated with the one or more segmented images 417, and/or generate a prediction associated with pupil morphology (e.g., pupil size, likelihood of pupil constriction, etc.) during the surgery. Determining the size of the pupil 420 may include determining one or more obstructed portions of the one or more segmented images 417 using the one or more segmentation masks 418. The obstruction classifier ML model 412 may use the segmentation masks 418, e.g., segmentations masks 418 of the palpebral fissure and pupil, to ascertain the presence of one or more obstructions within an image.

In one aspect using the palpebral fissure and pupil as an example, the obstruction classifier ML model 412 may establish the contours of the palpebral fissure and pupil by binarizing their respective segmentation masks and applying edge computations to the corresponding segmented image. Points along the contours of the palpebral fissure and pupil may be vectorized to enable the calculation of the Euclidean distance between these two vectors. The smallest computed Euclidean distance value is then compared against an obstruction threshold. If the distance is larger than the obstruction threshold, the obstruction classifier ML model 412 may determine the point associated with the pupil contour that is used for the calculation is unobstructed. This calculation may be repeated for multiple (e.g., all) points/coordinates associated with the contour of the pupil, allowing the obstruction classifier ML model 412 to determine obstructed and unobstructed regions of the pupil. If the determination results in no obstructed portions associated with the pupil, the size of the pupil size may be based upon the size of the pupil segmentation mask 418.

If one or more obstructions portions of the pupil are identified (i.e., when the minimum distance from such calculations is larger than a threshold distance), the obstruction classifier ML model 412 may generate an ellipse based upon the unobstructed regions of the pupil. The obstruction classifier ML model 412 may then generate a non-normalized pupil size based upon the area of the ellipse. The pupil size may include metrics associated with the eye and/or pupil, including one or more of an area of the pupil, an eccentricity of the pupil, a convex hull of the pupil, a major axis length of the pupil, a minor axis length of the pupil, and/or a contour path length of the pupil. FIG. 4E illustrates exemplary images 464A-D associated with generating the size of an obscured pupil. FIG. 4E visually depicts at least a portion of the just-described method of calculating the size of an obstructed pupil by the obstruction classifier ML model 412. Image 464A illustrates a pupil 465A obstructed by a surgical tool and/or eyelid 465B. Image 464B illustrates the obstruction classifier ML model 412 determining obstructed and unobstructed portions of the pupil 465A. Image 464C illustrates the obstruction classifier ML model 412 generating an ellipse 465C associated with the unobstructed portions of the pupil 465A. Image 464D illustrates the ellipse 465C fitted over the original image 464A to visually depict the size of the pupil were it unobstructed. The size ellipse 465C is used by the obstruction classifier ML model 412 to determine the non-normalized size of the pupil 465A.

As previously described, the size of the pupil may be normalized, e.g., to compensate for magnification of the pupil in the images of the surgical video images 414 and subsequent preprocessed images/images data 416 and segmented images/image data 418. To normalize the size of the pupil, the obstruction classifier ML model 412 may determine the size of a limbus of the eye using unobstructed portions of the limbus in the one or more segmented images of the segmented image data 418. This may include a similar process as just-described with respect to determining the size of the pupil based upon unobstructed portions of the pupil in the segmented images/image data 418.

Also as previously described, the size of the limbus region is physically fixed and cannot be significantly altered under normal circumstances during one or more phases of the surgical procedure, allowing the size of the limbus to be used as a reference when normalizing the size of the pupil. Accordingly, the obstruction classifier ML model 412 may normalize the size of the pupil using the size of the limbus to generate the pupil size 420, e.g., by diving the size of the limbus computed from a first surgical video image using the limbus segmentation mask, by the size of the limbus in a second image, and multiplying that value by the size of the pupil in the second image.

Returning yet again to FIG. 4A, the server 405 may generate at least one electronic notification 422 based upon the size of the pupil 420. The notification 422 may indicate the size of the pupil (e.g., by providing a metric associated with the pupil size 420), a recommendation of a pupil expansion device (e.g., determined by the server 405 based upon the (change in) size of the pupil and using a support vector machine ML model), a billing code associated with the ophthalmic surgical procedure, a prediction associated with the IFIS (e.g., that the patient associated with the pupil size 420 has IFIS, does not have IFIS, is at risk IFIS, may be taking a medication which causes IFIS, etc.), and/or any other suitable content. The notification may be and/or include an audio notification, a visual notification, a tactile notification, and/or any other suitable type of notification 422.

The server 405 may deliver the notification 422 to a user device (e.g., user device 115), e.g., via the network 410. In some aspects, the user device which receives the notification may be the same user device which provided the surgical video images 414, such as the surgical system 415. For example, as illustrated in FIG. 4A, the notification 422 may be provided on a display (e.g., display 240) of the surgical system 415 and indicate that an expansion device is required for the surgical procedure.

The server 405 may determine the size of the pupil 420 once (e.g., during a single phase of the surgical procedure), or may determine the size of the pupil more than once (e.g., the size of the pupil during multiple phases of the surgical procedure). In at least one aspect, the surgical video data may include at least a first phase and a second phase of the ophthalmic surgical procedure (e.g., when the entire ophthalmic surgical procedure is captured by the surgical video data). In such an aspect, the server 405 may determine the size of the pupil 420 during at least the first phase and second phases of the ophthalmic surgical procedure, and further determine (e.g., via the processor 402, models 406, 408, 412, or any other suitable component) a change in the size of the pupil based upon comparing the size of the pupil 420 during at least the first and second phases of the surgical procedure. When determining a change in size of the pupil. the size of the pupil 420 may also, or alternatively, indicate the change in size of the pupil.

In at least one aspect, the server 405 may determine the size of the pupil 420 in the surgical video data during the paracentesis, medication injection, viscoelastic insertion, main wound, capsulorrhexis initiation, capsulorrhexis formation, hydrodissection, phacoemulsification, cortical removal, lens insertion, viscoelastic removal, and/or wound closure phases of a cataract surgical procedure. Moreover, the notification 422 may further be based upon, and/or associated with, the change in the size of the pupil 420 during these phases of surgery. In one example, the notification 422 may indicate that the size of the pupil exceeds a threshold (e.g., by indicating the pupil size, change in size, provide a warning, etc.) such that an expansion device is recommended, and further indicate a billing code for a complex cataract surgical procedure based upon the use of the expansion device. In one example, after the medication injection phase when the pupil may be expected the dilate to at least a minimum size, the determination of the change in size of the pupil 420 may indicate the pupil has not dilated to the minimum size, and the server may generate a notification 422 indicating the surgeon should halt the surgical procedure unless the pupil further dilates.

Although in some embodiments one or more trained ML models are described as having specific functionality (e.g., the AWTFE ML model, anatomy segmentation ML model, and/or obstruction classifier ML model generating preprocessed image data, segmented image data/segmentation masks, and/or a pupil size respectively), in other embodiments the disclosed systems, methods and techniques may include fewer trained ML models (e.g., a single trained ML model) or additional ML models (e.g., four different ML models rather than three) having the same functionality of the ML models described. In one example, one ML model may generate the segmented image data, and another ML model may generate the segmented image masks. Additionally, although generating preprocess image data, segmented image data, segmentation masks, and/or a pupil size may be discussed in the context of cataract or ophthalmic surgical procedures, any other suitable type of surgical procedure and the like may be associated and/or the subject of one or more embodiments and/or aspects of the disclosed systems, methods and techniques.

### Exemplary Computer-Implemented Method for Determining Pupil Morphology During an Ophthalmic Surgical Procedure

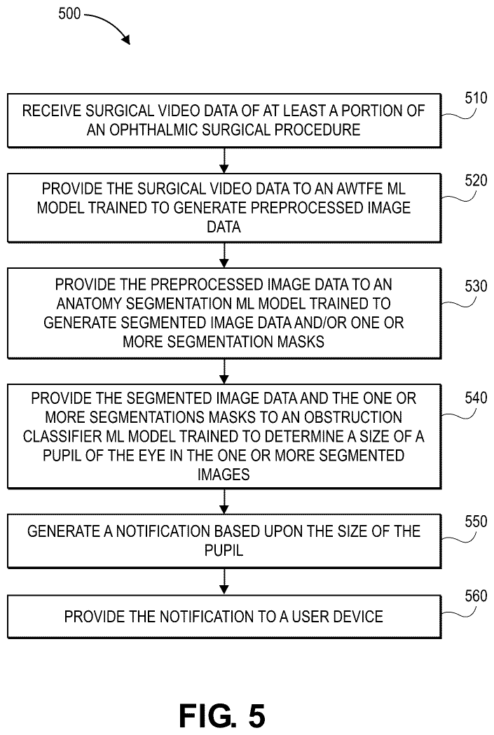

FIG. 5 is an exemplary block flow diagram depicting a computer-implemented method 500 for determining pupil morphology during an ophthalmic surgical procedure, such as a cataract surgery, a vitreoretinal surgery, or a corneal surgery. In general, the computer-implemented method 500 may be carried out by the devices, models, and/or other components of the computing environment 100 and/or the Pupil Morphology System 400. One or more steps of the computer-implemented method 500 may be implemented as a set of instructions stored on a computer-readable memory and executable by one or more processors. The computer-implemented method 500 of FIG. 5 may be implemented via one or more local or remote processors such as processor 120, servers such as servers 105, 405, user devices such as user devices 115, 215, and/or other electronic or electrical components, which may be in wired or wireless communication with one another.

According to an embodiment, the computer-implemented method 500 may include receiving, by one or more processors, surgical video data of at least a portion of an ophthalmic surgical procedure (block 510). The surgical video data may include one or more images of at least a portion of an eye.

The computer-implemented method 500 may include providing, by the one or more processors, the surgical video data to the AWTFE ML model trained to generate preprocessed image data (block 520). The AWTFE ML model may be trained using feature extraction training data, e.g., training data 320, historical surgical video data, historical processed image data, and/or any other suitable training data. The preprocessed image data may include one or more preprocessed images (e.g., color images) including extracted features associated with anatomical components of the eye, such as the pupil, the limbus, the sclera, the palpebral fissure, and/or other suitable anatomical component.

In at least one aspect of the computer-implemented method 500, to generate the preprocessed image data (block 520), the AWFTE ML model may be further trained to generate a third-order tensor of the pupil based upon a color image of the pupil of the surgical video data.

The computer-implemented method 500 may include providing, by the one or more processors, the preprocessed image data to an anatomy segmentation ML model trained to generate segmented image data and/or one or more segmentation masks (block 530). The anatomy segmentation ML model may be trained using anatomy segmentation training data, such as historical processed image data, historical segmented image data, historical segmentation masks, and/or any other suitable training data. In at least one aspect of the computer-implemented method 500, the anatomy segmentation ML model may include a CNN. The segmented image data may include one or more segmented images wherein the anatomical components are segmented. The one or more segmentation masks may be associated with the anatomical components. In at least one aspect of the computer-implemented method 500, the one or more segmentation masks are associated with one or more of a palpebral fissure, the limbus, or the pupil.

The computer-implemented method 500 may include providing, by the one or more processors, the segmented image data and the one or more segmentation masks to an obstruction classifier ML model trained to determine a size of a pupil of the eye in the one or more segmented images (block 540). The pupil size may include metrics associated with the eye including one or more of an area, an eccentricity, a convex hull, a major axis length, a minor axis length, or a contour path length. The anatomy segmentation ML model may be trained using obstruction classifier training data, such as historical segmented image data, historical segmentation masks, historical pupil image sizes, historical limbus sizes, and/or any other suitable training data. The determination of the size of the pupil (block 540) may include (i) determining, by the one or more processors, one or more obstructed portions of the one or more segmented images using the one or more segmentation masks; (ii) determining, by the one or more processors, the size of the pupil using unobstructed portions of the pupil in the one or more segmented images; (iii) determining, by the one or more processors, the size of a limbus of the eye using unobstructed portions of the limbus in the one or more segmented images; and (iv) normalizing, by the one or more processors, the size of the pupil using the size of the limbus. In at least one aspect of the computer-implemented method 500, the one or more obstructed portions of the one or more segmented images may include obstructions from one or more of a surgical instrument, an eyelid, or a surgical drape. In at least one aspect of the computer-implemented method 500, the size of the pupil may be normalized based upon magnification of the pupil in the one or more segmented images.

The computer-implemented method 500 may include generating, by the one or more processors, a notification based upon the size of the pupil (block 550). In at least one aspect of the computer-implemented method 500, the notification may indicate the size of the pupil, a recommendation of a pupil expansion device, a billing code associated with the ophthalmic surgical procedure, or a prediction associated with intraoperative floppy iris syndrome.

The computer-implemented method 500 may include providing, by the one or more processors, the notification to a user device (block 560). The notification may indicate the actual and/or predicted size and/or roundness of the pupil during one or more phases of surgery, the actual and/or predicted change in size of the pupil during various phases of surgery, a recommendation of a pupil expansion device (e.g., to use a pupil expansion device during one or more phases of surgery), development of iris billowing, development of entrapment of iris in incisions, a billing code associated with the ophthalmic surgical procedure (e.g., a complex or non-complex surgery billing code), a prediction associated with intraoperative floppy iris syndrome (e.g., that the subject of the surgery has intraoperative floppy iris syndrome, predictions of developing intraoperative floppy iris syndrome, is part of a drug trial to determine whether the drug causes intraoperative floppy iris syndrome, etc.), and/or any other suitable notification.

In at least one aspect of the computer-implemented method 500, the surgical video data may include at least a first phase and a second phase of the ophthalmic surgical procedure. In such an aspect, the computer-implemented method 500 may include (i) determining, by the one or more processors, the size of the pupil of the eye during the first phase of the ophthalmic surgical procedure; (ii) determining, by the one or more processors, the size of the pupil of the eye during the second phase of the ophthalmic surgical procedure; and (iii) determining, by the one or more processors, a change in the size of the pupil between the first phase and the second phase based upon comparing the size of the pupil during the first phase and the second phase. In such an aspect, generating the notification (block 550) may be further based upon the change in the size of the pupil.

It should be understood that not all blocks of the exemplary flow diagram of FIG. 5 are required to be performed.

### ADDITIONAL CONSIDERATIONS

With the foregoing, users whose data is being collected and/or utilized may first opt-in. After a user provides affirmative consent, data may be collected from the user's device (e.g., a mobile computing device). In other embodiments, deployment and use of ML models at a client or user device may have the benefit of removing any concerns of privacy or anonymity, by removing the need to send any personal or private data to a remote server.

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment”, “in one aspect” and/or the like in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory product to which it is communicatively coupled. A further hardware module may then, at a later time, access the memory product to retrieve and process the stored output. Hardware modules may also initiate communications with input or output products, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a building environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a building environment, an office environment, or a server farm). In other example embodiments, the one or more processors or processor-implemented modules may be distributed across a number of geographic locations.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Upon reading this disclosure, those of skill in the art will appreciate still additional alternative structural and functional designs for the method and systems described herein through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

Thus, many modifications and variations may be made in the techniques, methods, and structures described and illustrated herein without departing from the spirit and scope of the present claims. Accordingly, it should be understood that the methods and apparatus described herein are illustrative only and are not limiting upon the scope of the claims.

What is claimed:

1. A system for determining pupil morphology during an ophthalmic surgical procedure, the system comprising:
one or more processors; and
one or more non-transitory memories storing processor-executable instructions that, when executed by the one or more processors, cause the system to:
receive surgical video data of at least a portion of an ophthalmic surgical procedure, the surgical video data comprising one or more images of at least a portion of an eye;
provide the surgical video data to an adaptive wavelet tensor feature extraction machine learning model trained using feature extraction training data to generate preprocessed image data comprising one or more preprocessed images including extracted features associated with anatomical components of the eye;
provide the preprocessed image data to an anatomy segmentation machine learning model trained using anatomy segmentation training data to generate:
segmented image data comprising one or more segmented images wherein the anatomical components are segmented; and
one or more segmentation masks associated with the anatomical components;
provide the segmented image data and the one or more segmentation masks to an obstruction classifier machine learning model trained using obstruction classifier training data to determine a size of a pupil of the eye in the one or more segmented images, the determination of the size of the pupil comprising:
determining one or more obstructed portions of the one or more segmented images using the one or more segmentation masks;
determining the size of the pupil using unobstructed portions of the pupil in the one or more segmented images;
determining the size of a limbus of the eye using unobstructed portions of the limbus in the one or more segmented images; and
normalizing the size of the pupil using the size of the limbus;
generate a notification based upon the size of the pupil; and
provide the notification to a user device.

2. The system of claim 1, wherein the ophthalmic surgical procedure is selected from the group consisting of a cataract surgery, a vitreoretinal surgery, or a corneal surgery.

3. The system of claim 1, wherein the one or more images include color images.

4. The system of claim 1, wherein the anatomical components include one or more of the pupil, the limbus, a sclera, or a palpebral fissure.

5. The system of claim 1, wherein to generate the preprocessed image data, the adaptive wavelet tensor feature extraction machine learning model is further trained to generate a third-order tensor of the pupil based upon a color image of the pupil of the surgical video data.

6. The system of claim 1, wherein the anatomy segmentation machine learning model includes a convolutional neural network.

7. The system of claim 1, wherein the one or more segmentation masks are associated with one or more of a palpebral fissure, the limbus, or the pupil.

8. The system of claim 1, wherein the one or more obstructed portions of the one or more segmented images include obstructions from one or more of a surgical instrument, an eyelid, or a surgical drape.

9. The system of claim 1, wherein the notification indicates one or more of the size of the pupil, a prediction of pupil morphology, a recommendation of a pupil expansion device, a billing code associated with the ophthalmic surgical procedure, or a prediction associated with intraoperative floppy iris syndrome.

10. The system of claim 1, wherein the size of the pupil is normalized based upon magnification of the pupil in the one or more segmented images.

11. The system of claim 1, wherein the size of the pupil includes one or more of an area of the pupil, an eccentricity of the pupil, a convex hull of the pupil, a major axis length of the pupil, a minor axis length of the pupil, or a contour path length of the pupil.

12. The system of claim 1, wherein the surgical video data includes at least a first phase and a second phase of the ophthalmic surgical procedure, the system further comprising instructions that, when executed by the one or more processors, cause the system to:
determine the size of the pupil of the eye during the first phase of the ophthalmic surgical procedure; and
one or more of:
determine the size of the pupil of the eye during the second phase of the ophthalmic surgical procedure, and determine a change in the size of the pupil between the first phase and the second phase of the ophthalmic surgical procedure based upon comparing the size of the pupil during the first phase and the second phase of the ophthalmic surgical procedure, wherein generating the notification is further based upon the change in the size of the pupil, or
predict the size of the pupil of the eye during the second phase of the ophthalmic surgical procedure based upon the size of the pupil during the first phase of the ophthalmic surgical procedure, wherein generating the notification is further based upon the prediction of the size of the pupil.

13. The system of claim 12, wherein a phase of the ophthalmic surgical procedure is selected from the group consisting of paracentesis, medication injection, viscoelastic insertion, main wound, capsulorrhexis initiation, capsulorrhexis formation, hydrodissection, phacoemulsification, cortical removal, lens insertion, viscoelastic removal, and wound closure.

14. A computer-implemented method for determining pupil morphology during an ophthalmic surgical procedure, the computer-implemented method comprising:
receiving, by one or more processors, surgical video data of at least a portion of an ophthalmic surgical procedure, the surgical video data comprising one or more images of at least a portion of an eye;
providing, by the one or more processors, the surgical video data to an adaptive wavelet tensor feature extraction machine learning model trained using feature extraction training data to generate preprocessed image data comprising one or more preprocessed images including extracted features associated with anatomical components of the eye;
providing, by the one or more processors, the preprocessed image data to an anatomy segmentation machine learning model trained using anatomy segmentation training data to generate:
segmented image data comprising one or more segmented images wherein the anatomical components are segmented; and
one or more segmentation masks associated with the anatomical components;
providing, by the one or more processors, the segmented image data and the one or more segmentation masks to an obstruction classifier machine learning model trained using obstruction classifier training data to determine a size of a pupil of the eye in the one or more segmented images, the determination of the size of the pupil comprising:
determining, by the one or more processors, one or more obstructed portions of the one or more segmented images using the one or more segmentation masks;
determining, by the one or more processors, the size of the pupil using unobstructed portions of the pupil in the one or more segmented images;
determining, by the one or more processors, the size of a limbus of the eye using unobstructed portions of the limbus in the one or more segmented images; and
normalizing, by the one or more processors, the size of the pupil using the size of the limbus;
generating, by the one or more processors, a notification based upon the size of the pupil; and
providing, by the one or more processors, the notification to a user device.

15. The computer-implemented method of claim 14, wherein the ophthalmic surgical procedure is selected from the group consisting of a cataract surgery, a vitreoretinal surgery, or a corneal surgery.

16. The computer-implemented method of claim 14, wherein the anatomical components include one or more of the pupil, the limbus, a sclera, or a palpebral fissure.

17. The computer-implemented method of claim 14, wherein to generate the preprocessed image data, the adaptive wavelet tensor feature extraction machine learning model is further trained to generate a third-order tensor of the pupil based upon a color image of the pupil of the surgical video data.

18. The computer-implemented method of claim 14, wherein the one or more segmentation masks are associated with one or more of a palpebral fissure, the limbus, or the pupil.

19. The computer-implemented method of claim 14, wherein the surgical video data includes at least a first phase and a second phase of the ophthalmic surgical procedure, the computer-implemented method further comprising:
determining, by the one or more processors, the size of the pupil of the eye during the first phase of the ophthalmic surgical procedure; and
one or more of:
determining, by the one or more processors, the size of the pupil of the eye during the second phase of the ophthalmic surgical procedure, and determining, by the one or more processors, a change in the size of the pupil between the first phase and the second phase of the ophthalmic surgical procedure based upon comparing the size of the pupil during the first phase and the second phase of the ophthalmic surgical procedure, wherein generating the notification is further based upon the change in the size of the pupil, or
predicting, by the one or more processors, the size of the pupil of the eye during the second phase of the ophthalmic surgical procedure based upon the size of the pupil during the first phase of the ophthalmic surgical procedure, wherein generating the notification is further based upon the prediction of the size of the pupil.

20. A non-transitory computer-readable storage medium storing executable instructions that, when executed by a processor, cause a computer to at least:
receive surgical video data of at least a portion of an ophthalmic surgical procedure, the surgical video data comprising one or more images of at least a portion of an eye;
provide the surgical video data to an adaptive wavelet tensor feature extraction machine learning model trained using feature extraction training data to generate preprocessed image data comprising one or more preprocessed images including extracted features associated with anatomical components of the eye;
provide the preprocessed image data to an anatomy segmentation machine learning model trained using anatomy segmentation training data to generate:
segmented image data comprising one or more segmented images wherein the anatomical components are segmented; and
one or more segmentation masks associated with the anatomical components;
provide the segmented image data and the one or more segmentation masks to an obstruction classifier machine learning model trained using obstruction classifier training data to determine a size of a pupil of the eye in the one or more segmented images, the determination of the size of the pupil comprising:
determining one or more obstructed portions of the one or more segmented images using the one or more segmentation masks;
determining the size of the pupil using unobstructed portions of the pupil in the one or more segmented images;
determining the size of a limbus of the eye using unobstructed portions of the limbus in the one or more segmented images; and
normalizing the size of the pupil using the size of the limbus;
generate a notification based upon the size of the pupil; and
provide the notification to a user device.
